(19) United States
(12) Patent Application Publication
(10) Pub. No.: US 2012/0108529 A1
(43) Pub. Date: May 3, 2012

(54) PROTEASE INHIBITORS

(76) Inventors: Robert R. Webb, II, San Diego, CA (US); Mitchell W. Mutz, La Jolla, CA (US); Kenneth J. Barr, Boston, MA (US)

(21) Appl. No.: 13/319,681

(22) PCT Filed: May 14, 2010
(86) PCT No.: PCT/US10/35025

§ 371 Date: Jan. 13, 2012

Related U.S. Application Data
(60) Provisional application No. 61/178,771, filed on May 15, 2009.

(51) Int. Cl. A61K 31/706; C07D 211/60; C07D 401/14; C07D 498/18; C07H 15/203; C07D 497/04; A61K 31/4545; A61K 31/445; A61K 31/513; A61K 31/4725; A61K 31/436; A61K 31/4525; A61P 31/18; A61P 37/00; C12N 9/99; C07D 401/12

(52) U.S. Cl. 514/25; 546/194; 546/226; 544/316; 546/147; 540/456; 536/17.3; 546/197; 514/318; 514/330; 514/274; 514/307; 514/291; 514/321; 435/184

(57) ABSTRACT

Compounds useful as protease inhibitors are provided, as are methods of use and preparation of such compounds and compositions containing such compounds. In one embodiment, the compounds are useful for inhibiting HIV protease enzymes, and are therefore useful in slowing the proliferation of HIV.

### CROSS-REFERENCE TO RELATED APPLICATIONS

This application claims priority under 35 U.S.C. §119(e) to provisional U.S. application Ser. No. 61/178,771, filed May 15, 2009, the entire contents of which is incorporated herein by reference.

### TECHNICAL FIELD

The present disclosure relates to compounds useful for inhibiting protease enzymes, as well as methods of use and methods of manufacture of such compounds. The disclosure finds utility, for example, in the field of pharmacology.

### BACKGROUND

A wide range of diseases are caused by retroviruses. As presently understood, acquired immunodeficiency syndrome (AIDS) is a disease of the immune system caused by the retrovirus HIV (Human Immunodeficiency Virus). According to estimates from the World Health Organization, AIDS affects millions of people and is continuing to spread. In virtually all cases, AIDS causes a gradual breakdown of the body's immune system as well as progressive deterioration of the central and peripheral nervous systems.

The retroviral genome is composed of RNA which is converted to DNA by reverse transcription. This retroviral DNA is then stably integrated into a host cell's chromosome and, employing the replicative processes of the host cells, produces new retroviral particles and advances the infection to other cells. HIV appears to have a particular affinity for the human T-4 lymphocyte cell which plays a vital role in the body's immune system. HIV infection of these white blood cells depletes this white cell population. Eventually, the immune system is rendered inoperative and ineffective against various opportunistic diseases such as, among others, pneumocystic carinii pneumonia, Kaposi's sarcoma, and cancer of the lymph system.

Retroviral replication routinely features post-translational processing of polyproteins. This yields mature polypeptides that will subsequently aid in the formation and function of infectious virus. In the case of HIV, this post-translational processing is accomplished by virally encoded HIV protease enzyme. A retroviral protease is a proteolytic enzyme that participates in the maturation of new infectious virions in infected cells during the reproductive cycle. Interruption of the normal viral reproduction cycle can be affected by disrupting the protease enzyme. Therefore, inhibitors of HIV protease may function as anti-HIV viral agents.

On-going treatment of HIV-infected individuals with compounds that inhibit HIV protease has led to the development of mutant viruses that possess proteases that are resistant to the inhibitory effect of these compounds. Thus, to be effective, it is desirable that new HIV protease inhibitors are effective not only against wild-type strains of HIV, but also against the newly emerging mutant strains that are resistant to the commercially available protease inhibitors.

Some antiviral compounds that act as HIV protease inhibitors are described in WO 99/67254. Known HIV protease inhibitors include: saquinavir; ritonavir; indinavir; nelfinavir; amprenavir; lopinavir; atazanavir; fosamprenavir; tipranavir; and darunavir.

In addition to the problematic development of strains of the virus resistant to known inhibitors, some HIV protease inhibitors are difficult to prepare, are expensive to obtain, and/or have significant adverse side effects; all of these drawbacks may result in lower patient compliance and less effective treatment. Accordingly, there continues to be a need for the development of new inhibitors effective to inhibit the HIV protease in both wild type and mutant strains of HIV.

### SUMMARY OF THE DISCLOSURE

The present disclosure provides compounds that address one or more of the abovementioned drawbacks. In particular, the present disclosure provides compounds useful as protease inhibitors.

In some embodiments, the disclosure provides compounds having the structure of formula (I)

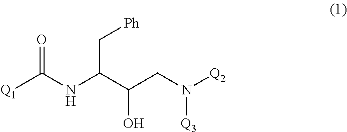

wherein:

Q¹ is substituted heteroatom-containing alkyl, optionally substituted with -L-U, or Q¹ is cycloalkoxy;

Q² is arylsulfonyl substituted with -L-U, or Q² is alkylamido optionally substituted with -L-U, provided that either Q¹ or Q² is substituted with -L-U;

Q³ is alkyl or aralkyl, or wherein Q² and Q³ are taken together to form a cyclic group;

L is a linking moiety;

U is selected from Unit A, Unit B, and Unit C

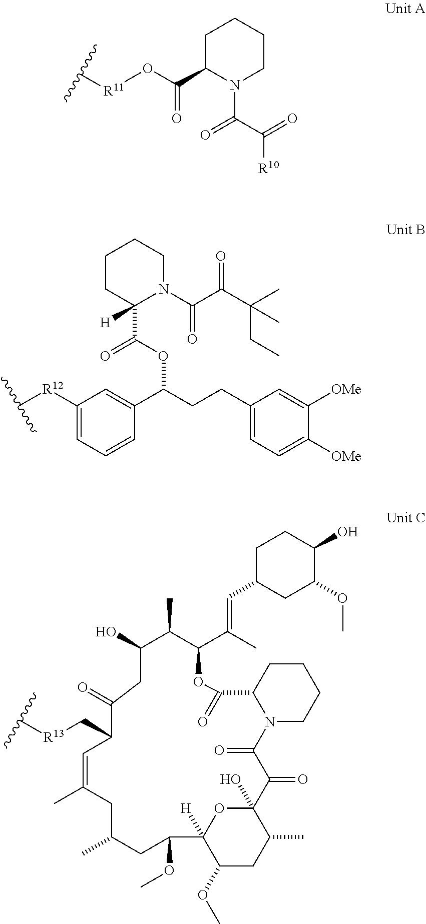

wherein:

the wavy line represents the attachment point to the remainder of the compound;

R¹⁰ is selected from

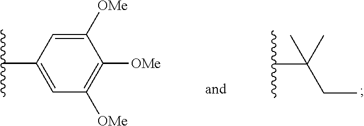

R¹¹ is selected from a bond, —(CH₂)ₙ₁—, —(CH₂)ₙ₁—O—, and —(CH₂)ₙ₁—NH—, where n1 is an integer from 1 to 5 (that is, 1, 2, 3, 4, or 5);

R¹² is selected from a bond, —(CH₂)ₙ₂—, —(CH₂)ₙ₂—O—, and —O—(CH₂)ₙ₂—NH—, where n2 is an integer from 1 to 5 (that is, 1, 2, 3, 4, or 5); and

R¹³ is selected from a bond, —(CH₂)ₙ₃—, —(CH₂)ₙ₃—O—, and —(CH₂)ₙ₃—NH—, where n3 is an integer from 1 to 5 (that is, 1, 2, 3, 4, or 5), or a stereoisomer, salt, or prodrug thereof.

In a further embodiment, there is provided compounds having a structure selected from formulae (II), (III), (IVa), and (IVb)

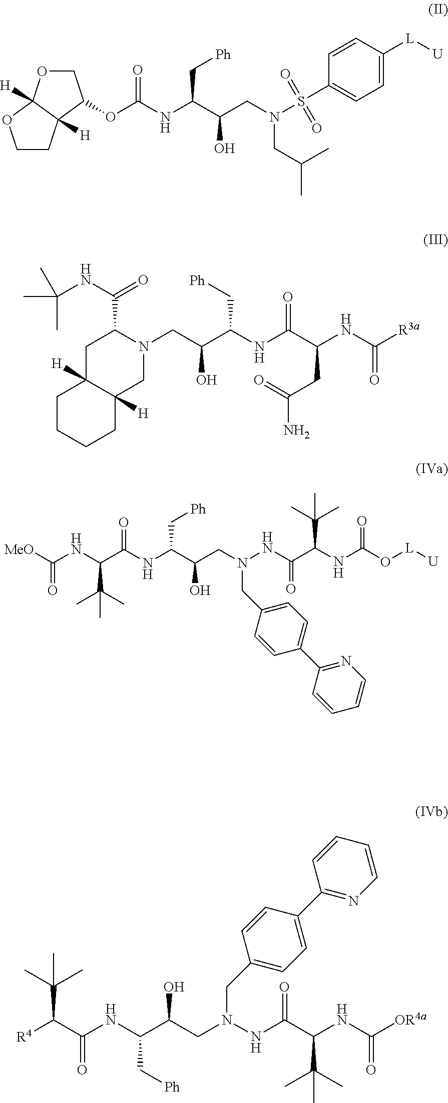

wherein:

R^{3a} is selected from

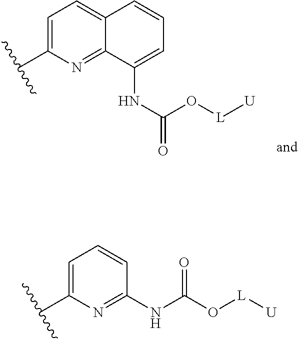

R⁴ is selected from —NH—C(═O)—O-L-U and -L-U;

R^{4a} is selected from alkyl, aryl, alkaryl, and aralkyl; and

L and U are as defined for Formula (I).

In still further embodiments, there is provided a method for inhibiting the action of HIV-1 protease, the method comprising administering a conjugate compound comprising: (1) a core selected from atazanavir, saquinavir, darunavir, and analogs or derivatives thereof; (2) a linking moiety; and (3) a second moiety capable of binding to a FK506-binding protein.

Further embodiments will be apparent from the disclosure provided, including the examples and claims.

### DETAILED DESCRIPTION OF THE INVENTION

Unless otherwise indicated, the disclosure is not limited to specific procedures, starting materials, or the like, as such may vary. It is also to be understood that the terminology used herein is for the purpose of describing particular embodiments only and is not intended to be limiting.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

The term “alkyl” as used herein refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C₁-C₆ alkoxy” or “lower alkoxy” herein may, for example, may contain 1 to 3 carbon atoms, and as a further example, such substituents may contain 1 or 2 carbon atoms (i.e., methoxy and ethoxy).

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

The term “alkylene” as used herein refers to a di-radical alkyl group. Unless otherwise indicated, such groups include saturated hydrocarbon chains containing from 1 to 24 carbon atoms, which may be substituted or unsubstituted, may contain one or more alicyclic groups, and may be heteroatom-containing. “Lower alkylene” refers to alkylene linkages containing from 1 to 6 carbon atoms. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

Similarly, the terms “alkenylene,” “alkynylene,” “arylene,” “aralkylene,” and “alkarylene” as used herein refer to di-radical alkenyl, alkynyl, aryl, aralkyl, and alkaryl groups, respectively.

The term “amino” is used herein to refer to the group —NZ¹Z² wherein Z¹ and Z² are hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including unsubstituted, substituted and/or heteroatom-containing hydrocarbyl moieties.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation: functional groups such as halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(O)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-substituted C₁-C₂₄ alkylcarbamoyl (—(CO)—NH(C₁-C₂₄ alkyl)), di-substituted alkylcarbamoyl (—(CO)—N(C₁-C₂₄ alkyl)₂), mono-substituted arylcarbamoyl (—(CO)—NH-aryl), thiocarbamoyl (—(CS)—NH₂), carbamide (—NH—(CO)—NH₂), cyano (—C≡N), isocyano (—N⁺≡C⁻), cyanato (—O≡N), isocyanate (—O—N⁺≡C⁻), isothiocyanato (—S—C≡N), azido (—N═N⁺≡N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₅-C₂₀ arylamido (—NH—(CO)-aryl), imino (—CR═NH where R=hydrogen, C₁-C₂₄ alkyl, C₅-C₂₀ aryl, C₆-C₂₀ alkaryl, C₆-C₂₀ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH⁻¹)₂), phosphonato (—P(O)(O⁻)₂), phosphinato (—P(O)(O⁻)), phospho (—PO₂), and phosphino (—PH₂), mono- and di-(C₁-C₂₄ alkyl)-substituted phosphino, mono- and di-(C₅-C₂₀ aryl)-substituted phosphino; and the hydrocarbyl moieties C₁-C₂₄ alkyl (including C₁-C₁₈ alkyl, further including C₁-C₁₂ alkyl, and further including C₁-C₆ alkyl), C₂-C₂₄ alkenyl (including C₂-C₁₈ alkenyl, further including C₂-C₁₂ alkenyl, and further including C₂-C₆ alkenyl), C₂-C₂₄ alkynyl (including C₂-C₁₈ alkynyl, further including C₂-C₁₂ alkynyl, and further including C₂-C₆ alkynyl), C₅-C₃₀ aryl (including C₅-C₂₀ aryl, and further including C₅-C₁₂ aryl), and C₆-C₃₀ aralkyl (including C₆-C₂₀ aralkyl, and further including C₆-C₁₂ aralkyl). In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated. Furthermore, the above groups may be used as a linking moiety where appropriate (e.g., C₂-C₂₄ alkylcarbonato can be C₂-C₂₄ alkylene-carbonato (also denoted —O—(CO)—O-alkyl-), C₅-C₂₀ arylsulfonyl can be C₅-C₂₀ arylene-sulfonyl (also denoted —SO₂-arylene-), etc.).

When the term “substituted” appears prior to a list of possible substituted groups, it is intended that the term apply to every member of that group. For example, the phrase “substituted alkyl and aryl” is to be interpreted as “substituted alkyl and substituted aryl.”

Unless otherwise indicated, the terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, the terms include prophylactic use of active agents. “Preventing” a disorder or unwanted physiological event in a patient refers specifically to the prevention of the occurrence of symptoms and/or their underlying cause, wherein the patient may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the individual, the particular beneficial agent or agents, and the like. As used herein, and unless specifically stated otherwise, an “effective amount” of a beneficial refers to an amount covering both therapeutically effective amounts and prophylactically effective amounts.

As used herein, a “therapeutically effective amount” of an active agent refers to an amount that is effective to achieve a desired therapeutic result, and a “prophylactically effective amount” of an active agent refers to an amount that is effective to prevent or lessen the severity of an unwanted physiological condition. Therapeutically effective and prophylactically effective amounts of a given active agent will typically vary with respect to factors such as the type and severity of the disorder or disease being treated and the age, gender, and weight of the patient.

By a “pharmaceutically acceptable” component is meant a component that is not biologically or otherwise undesirable, i.e., the component may be incorporated into a pharmaceutical formulation of the disclosure and administered to a patient as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “pharmacologically active” (or simply “active”), as in a “pharmacologically active” derivative or analog, refers to a derivative or analog (e.g., a salt, ester, amide, conjugate, metabolite, isomer, fragment, etc.) having the same type of pharmacological activity as the parent compound and approximately equivalent in degree.

The term “controlled release” refers to a formulation, dosage form, or region thereof from which release of a beneficial agent is not immediate, i.e., with a “controlled release” dosage form, administration does not result in immediate release of the beneficial agent in an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release and delayed release formulations.

The term “sustained release” (synonymous with “extended release”) is used in its conventional sense to refer to a formulation, dosage form, or region thereof that provides for gradual release of a beneficial agent over an extended period of time, and that preferably, although not necessarily, results in substantially constant blood levels of the agent over an extended time period.

The term “naturally occurring” refers to a compound or composition that occurs in nature, regardless of whether the compound or composition has been isolated from a natural source or chemically synthesized.

As used herein, the term “protease inhibitor” refers to compounds that inhibit proteases of viral origin, and that are useful in the treatment of viral infections caused by retroviruses, such as HIV, in mammals, both human and nonhuman.

Certain compounds are referred to herein by common names. It is to be understood that, unless otherwise specified, such references include the named compound as well as analogs and derivatives thereof. For example, the term “FK506” refers to the parent compound as well as analogs and derivatives of FK506. Furthermore, the term “core” refers to a moiety comprising a portion of a molecule. For example, an “atazanavir core” is a moiety comprising a portion of the atazanavir molecular structure.

The disclosure provides the compounds shown and described in the Examples provided below. The disclosure further provides pharmaceutical formulations comprising such compounds, as well as methods of treatment using such compounds and formulations.

In some embodiments, the disclosure provides conjugates of any of the following core compounds: atazanavir; amprenavir; lopinavir; saquinavir; darunavir, or derivatives or analogs thereof. The conjugates comprise the core compound and a second moiety linked to the core compound through a linker moiety.

In some embodiments, the second moiety (herein also identified by the label “U”) is selected from the following structures:

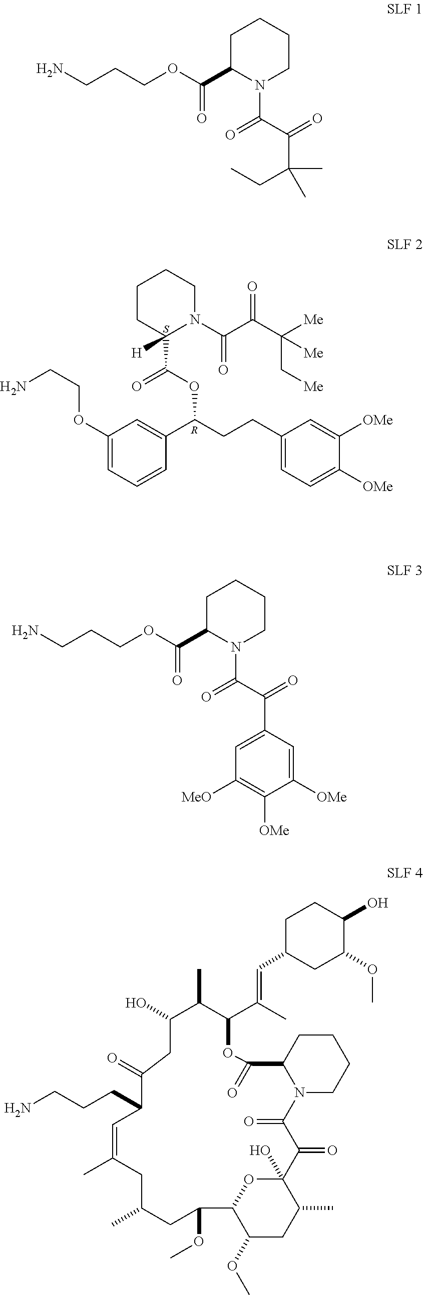

SLF 4 may also be referred to herein as FK506. It will be appreciated that the above core compounds and second moieties, when present in the conjugates disclosed herein, will be present as radical species—i.e., one atom or group (typically, although not necessarily, H) removed to accommodate the linkage to the remainder of the conjugate compound.

In some embodiments, the linker moiety (herein also identified by the label “L”) is selected from the following moieties:

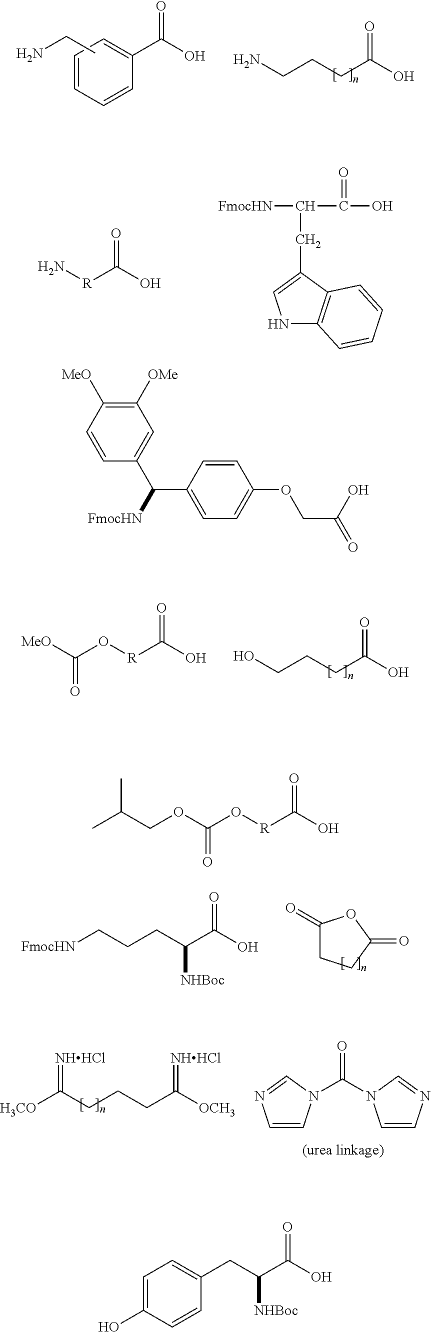

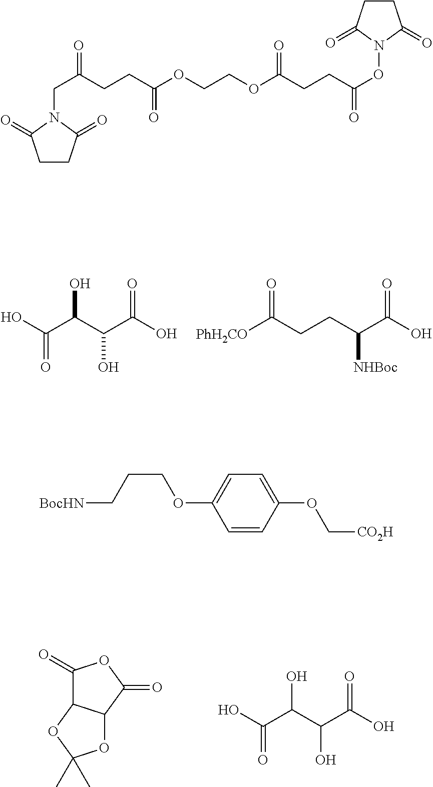

wherein R is a hydrocarbyl group and n is an integer from 0 to 12, or from 1 to 8, or from 1 to 6, or from 1 to 4. Examples of linkers also include amino acid and substituted amino acids. Again it will be appreciated that the linker moieties shown above will be in modified form when incorporated into the conjugates of the disclosure. For example, the linkers may be in the form of di-radicals (i.e., two atoms or groups removed to accommodate linkage to the core compound and the second compound). Also for example, the linkers may be ring-opened form, or deprotected form.

The compounds described herein may further be modified as described in PCT application PCT/US06/43400, which is entitled “Improving the Pharmacokinetics of Protease Inhibitors and Other Drugs,” and which published as WO 2007/053792, the contents of which are incorporated herein by reference.

In some embodiments, then, the compounds of the invention have the structure of formula (I)

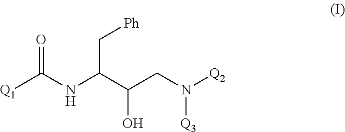

wherein:

Q¹ is substituted heteroatom-containing alkyl optionally substituted with -L-U, or Q¹ is cycloalkoxy;

Q² is arylsulfonyl substituted with -L-U, or Q² is alkylamido optionally substituted with -L-U, provided that either Q¹ or Q² is substituted with -L-U;

Q³ is alkyl or aralkyl, or wherein Q² and Q³ are taken together to form a cyclic group;

L is a linking moiety as described herein; and

U is a second moiety as described herein. For example, U can be Unit A, Unit B, or Unit C, which are described in more detail below.

Stereoisomers (e.g., enantiomers and diasteriomers), salts, or prodrugs of compounds having the structure of formula (I) are also within scope of the invention.

For example, Q¹ may be selected from —CR^{a}—X⁴, —O—Y¹, and —CR^{b}—NH—C(═O)—Z¹—NH—C(═O)—O-L-U. In some embodiments, Q¹ is alkoxyl (including cycloalkoxyl). In some embodiments, Q¹ is other than —O—(C₄H₇O) (i.e., —O-tetrahydrofuran-2-yl).

Also for example, Q² may be selected from —NH—C(═O)—CR^{a}—NH—C(═O)—O—X⁵ and —SO₂—Ar¹-L-U.

Also for example, Q³ may be selected from —CH₂—Ar¹—Ar² and alkyl, including substituted alkyl such as branched alkyl. In some embodiments, Q³ is selected from methyl, ethyl, propyl (including n-propyl and i-propyl), butyl (including n-butyl, sec-butyl, i-butyl, and t-butyl), pentyl, and hexyl.

In some embodiments, Q² and Q³ are linked to form a cycle. For example, in some embodiments, Q² and Q³, together with the nitrogen to which they are attached, form a five- or six-membered ring that may be alicyclic or aromatic, may be unsubstituted or substituted, and may contain one or more additional heteroatoms. In some embodiments, the cycle is part of a ring system that comprises two or more fused cycles. For example, in some embodiments, the cycle is alicyclic and comprises two fused six-member rings that may contain further substitution (including, for example, alkyl and substituted alkyl substituents).

Furthermore, X⁴ is an amide, a carbamate (i.e., —NH—(C═O)—OR, where R is selected from alkyl), or -L-U. In some embodiments, X⁴ is —NH—C(═O)—OCH₃.

Furthermore, X⁵ is alkyl, aryl, aralkyl, alkaryl, or -L-U. In some embodiments, X⁵ is methyl or substituted methyl, ethyl or substituted ethyl, propyl or substituted propyl, phenyl or substituted phenyl, benzyl or substituted benzyl.

Furthermore, Y¹ is a heterocyclic group. In some embodiments, Y¹ comprises two or more fused rings and two or more heteroatoms. For example, in some embodiments, Y¹ comprises two fused rings and two oxygen atoms, such as bis(tetrahydrofuranyl) or substituted versions thereof. In some embodiments, such as when Q² is —SO₂—Ar¹-L-U and Q³ is alkyl, Y¹ is not tetrahydrofuranyl.

Furthermore, Z¹ is an arylene group which may be substitute or unsubstituted, and which may contain one or more heteroatoms. For example, Z¹ may be a nitrogen-containing aryl group comprising one or more rings. In some embodiments, Z¹ is pyridylene (i.e., a pyridyl linking moiety) or quinolinylene (i.e., a quinolinyl linking moiety), such as 2,6-pyridylene, 2,5-pyridylene, 2,7-quinolinylene, or 2,8-quinolinylene. Other examples of Z¹ include phenylene, pyrimidylene, etc.

Furthermore, Ar¹ is an optionally substituted phenylene. For example, Ar¹ is 1,4-arylene, or 1,3-arylene, or 1,2-arylene, or any substituted version thereof.

Furthermore, Ar² is optionally substituted pyridyl. For example, Ar² is 2-pyridyl, or 3-pyridyl, or 4-pyridyl.

Furthermore, R^{a} is alkyl, including substituted alkyl such as branched alkyl. For example, R^{a} is selected from methyl, ethyl, propyl (including n-propyl and i-propyl), butyl (including n-butyl, sec-butyl, i-butyl, and t-butyl), pentyl, and hexyl. In some embodiments, R^{a} is t-butyl.

Furthermore, R^{b} is carbamoyl-substituted alkyl. In some embodiments, R^{b} is alkyl substituted with an alkylcarbamoyl or arylcarbamoyl. In some embodiments, R^{b} is alkyl substituted with an unsubstituted carbamoyl. For example, in some embodiments, R^{b} is —CH₂—C(═O)—NH₂.

In Formula (I), all possible stereoisomers are within the scope of the invention.

In some embodiments, the compounds of the invention have the structure of formula (Ia), (Ib), or (Ic)

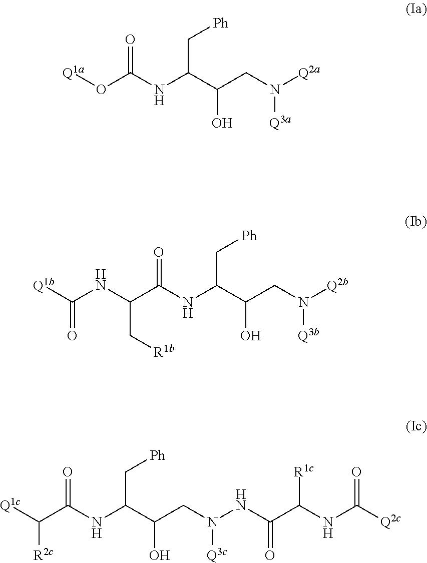

wherein the following definitions apply to Formulae (Ia), (Ib), or (Ic).

Q^{1a} is a cyclic group optionally comprising two or more fused rings and optionally heteroatom-containing. In preferred embodiments, Q^{1a} comprises two fused rings and two heteroatoms, such as two oxygen atoms, and is optionally further substituted.

Q^{2a} is —SO₂—Ar¹-L-U, wherein Ar¹ is an optionally substituted phenylene.

Q^{3a} is alkyl, including substituted alkyl, such as methyl, ethyl, propyl, and butyl.

Q^{1b} is —Z¹—NH—C(═O)-L-U, where Z¹ is as described above.

Q^{2b} and Q^{3b} are linked, together with the nitrogen atom to which they are attached, to form a heterocyclic ring system which optionally comprises 2 or more fused rings.

R^{1b} is substituted or unsubstituted carbamoyl.

Q^{1c} is selected from -L-U, alkylamido, and —NH—C(═O)—O-L-U.

Q^{2c} is selected from —O-L-U and —O—R^{3c}.

Q^{3c} is aralkyl which is optionally heteroatom-containing.

R^{1c} and R^{2c} are individually selected from alkyl groups.

R^{3c} is selected from alkyl, aryl, alkaryl, and aralkyl.

In Formulae (Ia), (Ib), and (Ic), all possible stereoisomers are within the scope of the invention

In some embodiments, the compounds of the invention have the structure of formula (II), (III), (IVa), or (IVb)

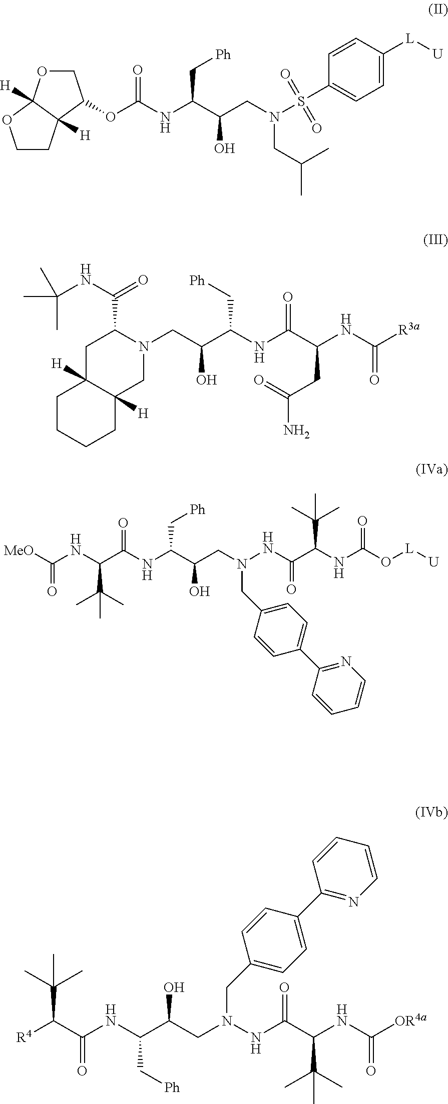

wherein:

R^{1a} is selected from

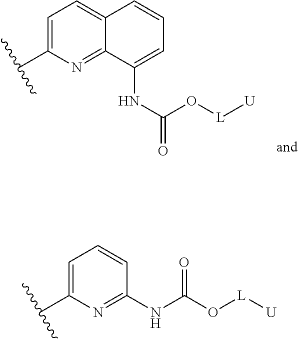

R⁴ is selected from —NH—C(═O)—O-L-U and -L-U;

R^{4a} is selected from alkyl, aryl, alkaryl, and alkaryl;

L is a linking group as defined herein; and

U is a second moiety as defined herein. Again, it will be appreciated that although certain stereochemical arrangements are shown in structures (II), (III), (IVa), and (IVb), the invention is not so limited.

Some embodiments of L, the linker moiety, are shown above. Generally, the linker moiety is selected from a bond, alkylene, alkenylene, alkynylene, arylene, aralkylene, and alkarylene, any of which may be substituted or unsubstituted, and any of which may contain one or more heteroatoms. More specific, some examples of linking moieties are substituted or unsubstituted heteroalkylene, heteroarylene, alkylenecarbonyl, arylenecarbonyl, alkyleneoxycarbonyl, aryleneoxycarbonyl, alkylenecarbonato, arylenecarbonato, alkylenecarbamoyl, arylcarbamoyl, alkyleneamine, aryleneamine, alkyleneamide, and aryleneamide.

Specific examples of linker moieties include the following: —(CH₂)ₙ—; —(CH₂)ₘ—C(═O)—; —(CH₂)ₙ—CH═CH—; —(CH₂)ₘ—O—C(═O)—; —(CH₂)ₙ—CH═CH—C(═O)—; —(CH₂)ₘ—CH═CH—C(═O)—(CH₂)ₙ—; and —(CH₂)ₙ—O—(CH₂)ₘ—C(═O)—, wherein n is an integer selected from 1, 2, 3, 4, 5, 6, 7, 8, 9, 10, 11, and 12, and wherein m is an integer selected from 0, 1, 2, 3, 4, 5, 6, 7, 8, 9, 10, 11, and 12. It will be appreciated that these and the linker moieties shown above may be incorporated into the compounds of the invention in any orientation (i.e., as written, or reversed).

Some embodiments of U, the second moiety, are provided above. In general, the second moiety is FK506, a derivative or analog thereof, or another synthetic ligand of FK506 binding proteins (“SLF”). Further examples include Unit A, Unit B, and Unit C as shown below:

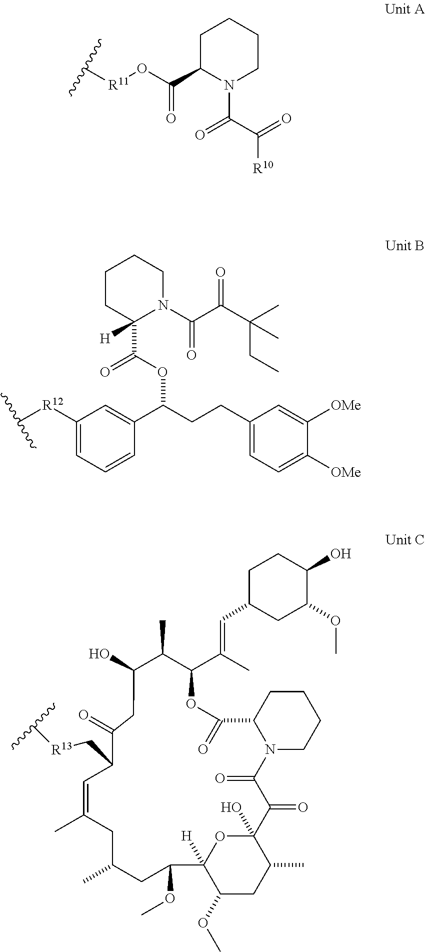

wherein:

the wavy line represents the attachment point to the remainder of the compound (i.e., either the linking moiety when present or to Formula (I));

R¹⁰ is selected from

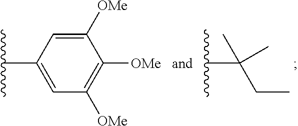

R¹¹ is selected from a bond, —(CH₂)ₙ₁—, —(CH₂)ₙ₁—O—, and —(CH₂)ₙ₁—NH—, where n1 is an integer from 1 to 5 (that is, 1, 2, 3, 4, or 5);

R¹² is selected from a bond, —(CH₂)ₙ₂—, —(CH₂)ₙ₂—O—, and —O—(CH₂)ₙ₂—NH—, where n2 is an integer from 1 to 5 (that is, 1, 2, 3, 4, or 5); and

R¹³ is selected from a bond, —(CH₂)ₙ₃—, —(CH₂)ₙ₃—O—, and —(CH₂)ₙ₃—NH—, where n3 is an integer from 1 to 5 (that is, 1, 2, 3, 4, or 5).

As with all linkers described herein, it will be appreciated that R¹¹, R¹², and R¹³ can be present as written or “reversed” (i.e., —(CH₂)ₙ₁—O— or —O—(CH₂)ₙ₁—).

In some embodiments of Unit A, R¹¹ is —(CH₂)ₙ₁—NH— and n1 is 2. In some embodiments of Unit B, R¹² is —(CH₂)ₙ₁—O— and n2 is 2. In some embodiments of Unit C, R¹³ is —(CH₂)ₙ₃—NH— and n3 is 2.

Where a compound of the invention can exist as stereoisomers, the formulations of the invention may comprise a single stereoisomer of such compound, or a mixture of stereoisomers of such compound. For example, where a compound has a single stereocenter, and thus may exist as one of two enantiomers, the formulations of the invention may comprise either of the two enantiomers in substantially pure form, or may comprise a mixture of the two enantiomers in any proportion (such as a 90/10 mixture, or a 80/20 mixture, or a 70/30 mixture, or a 60/40 mixture, or a 50/50 racemic mixture). For example, compounds having the structure of Formula (I) may exist as any of the four stereoisomers shown below (or combinations thereof), and it will be appreciated that each such stereoisomer is within the scope of the invention:

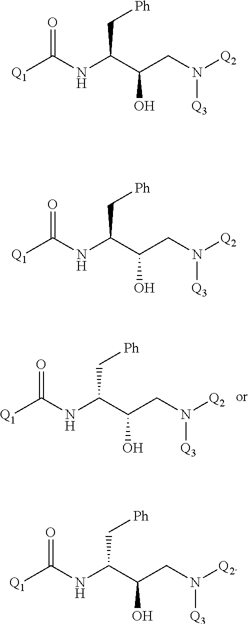

A compound of the disclosure may be administered in the form of a salt, ester, amide, prodrug, active metabolite, analog, or the like, provided that the salt, ester, amide, prodrug, active metabolite or analog is pharmaceutically acceptable and pharmacologically active in the present context. Salts, esters, amides, prodrugs, active metabolites, analogs, and other derivatives of the active agents may be prepared using standard procedures known to those skilled in the art of synthetic organic chemistry and described, for example, by J. March, Advanced Organic Chemistry: Reactions, Mechanisms and Structure, 5th Ed. (New York: Wiley-Interscience, 2001). Furthermore, where appropriate, functional groups on the compounds of the disclosure may be protected from undesired reactions during preparation or administration using protecting group chemistry. Suitable protecting groups are described, for example, in Green, Protective Groups in Organic Synthesis, 3rd Ed. (New York: Wiley-Interscience, 1999).

For example, where appropriate, any of the compounds described herein may be in the form of a pharmaceutically acceptable salt. A pharmaceutically acceptable salt may be prepared from any pharmaceutically acceptable organic acid or base, any pharmaceutically acceptable inorganic acid or base, or combinations thereof. The acid or base used to prepare the salt may be naturally occurring.

Suitable organic acids for preparing acid addition salts include, e.g., C₁-C₆ alkyl and C₆-C₁₂ aryl carboxylic acids, di-carboxylic acids, and tri-carboxylic acids such as acetic acid, propionic acid, succinic acid, maleic acid, fumaric acid, tartaric acid, glycolic acid, citric acid, pyruvic acid, oxalic acid, malic acid, malonic acid, benzoic acid, cinnamic acid, mandelic acid, salicylic acid, phthalic acid, and terephthalic acid, and aryl and alkyl sulfonic acids such as methanesulfonic acid, ethanesulfonic acid, and p-toluenesulfonic acid, and the like. Suitable inorganic acids for preparing acid addition salts include, e.g., hydrochloric acid, hydrobromic acid, hydroiodic acid, sulfuric acid, nitric acid, and phosphoric acid, and the like. An acid addition salt may be reconverted to the free base by treatment with a suitable base.

Suitable organic bases for preparing basic addition salts include, e.g., primary, secondary and tertiary amines, such as trimethylamine, triethylamine, tripropylamine, N,N-dibenzylethylenediamine, 2-dimethylaminoethanol, ethanolamine, ethylenediamine, glucamine, glucosamine, histidine, and polyamine resins, cyclic amines such as caffeine, N-ethylmorpholine, N-ethylpiperidine, and purine, and salts of amines such as betaine, choline, and procaine, and the like. Suitable inorganic bases for preparing basic addition salts include, e.g., salts derived from sodium, potassium, ammonium, calcium, ferric, ferrous, aluminum, lithium, magnesium, or zinc such as sodium hydroxide, potassium hydroxide, calcium carbonate, sodium carbonate, and potassium carbonate, and the like. A basic addition salt may be reconverted to the free acid by treatment with a suitable acid.

Preparation of esters involves transformation of a carboxylic acid group via a conventional esterification reaction involving nucleophilic attack of an RO⁻ moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

Any of the compounds of the disclosure may be the active agent in a formulation as described herein. Formulations containing the compounds of the disclosure may include 1, 2, 3 or more of the compounds described herein, and may also include one or more additional active agents such as analgesics, antibiotics, and other anti-retroviral agents (such as reverse transcriptase inhibitors including 3′-azido-2′,3′-dideoxythymidine (AZT), 2′3′-dideoxycytidine (ddC), and 2′3′-dideoxyinosine (ddI)).

The amount of active agent in the formulation typically ranges from about 0.05 wt % to about 95 wt % based on the total weight of the formulation. For example, the amount of active agent may range from about 0.05 wt % to about 50 wt %, or from about 0.1 wt % to about 25 wt %. Alternatively, the amount of active agent in the formulation may be measured so as to achieve a desired dose.

Formulations containing the compounds of the disclosure may be presented in unit dose form or in multi-dose containers with an optional preservative to increase shelf life.

The compositions of the disclosure may be administered to the patient by any appropriate method. In general, both systemic and localized methods of administration are acceptable. It will be obvious to those skilled in the art that the selection of a method of administration will be influenced by a number of factors, such as the condition being treated, frequency of administration, dosage level, and the wants and needs of the patient. For example, certain methods may be better suited for rapid delivery of high doses of active agent, while other methods may be better suited for slow, steady delivery of active agent. Examples of methods of administration that are suitable for delivery of the compounds of the disclosure include parental and transmembrane absorption (including delivery via the digestive and respiratory tracts). Formulations suitable for delivery via these methods are well known in the art.

For example, formulations containing the compounds of the disclosure may be administered parenterally, such as via intravenous, subcutaneous, intraperitoneal, or intramuscular injection, using bolus injection and/or continuous infusion. Generally, parenteral administration employs liquid formulations.

The compositions may also be administered via the digestive tract, including orally and rectally. Examples of formulations that are appropriate for administration via the digestive tract include tablets, capsules, pastilles, chewing gum, aqueous solutions, and suppositories.

The formulations may also be administered via transmucosal administration. Transmucosal delivery includes delivery via the oral (including buccal and sublingual), nasal, vaginal, and rectal mucosal membranes. Formulations suitable for transmucosal deliver are well known in the art and include tablets, chewing gums, mouthwashes, lozenges, suppositories, gels, creams, liquids, and pastes.

The formulations may also be administered transdermally. Transdermal delivery may be accomplished using, for example, topically applied creams, liquids, pastes, gels and the like as well as what is often referred to as transdermal “patches.”

The formulations may also be administered via the respiratory tract. Pulmonary delivery may be accomplished via oral or nasal inhalation, using aerosols, dry powders, liquid formulations, or the like. Aerosol inhalers and imitation cigarettes are examples of pulmonary dosage forms.

Liquid formulations include solutions, suspensions, and emulsions. For example, solutions may be aqueous solutions of the active agent and may include one or more of propylene glycol, polyethylene glycol, and the like. Aqueous suspensions can be made by dispersing the finely divided active agent in water with viscous material, such as natural or synthetic gums, resins, methylcellulose, sodium carboxymethylcellulose, or other well known suspending agents. Also included are formulations of solid form which are intended to be converted, shortly before use, to liquid form.

Tablets and lozenges may comprise, for example, a flavored base such as compressed lactose, sucrose and acacia or tragacanth and an effective amount of an active agent. Pastilles generally comprise the active agent in an inert base such as gelatin and glycerine or sucrose and acacia. Mouthwashes generally comprise the active agent in a suitable liquid carrier.

For topical administration to the epidermis the chemical compound according to the disclosure may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Transdermal patches typically comprise: (1) a impermeable backing layer which may be made up of any of a wide variety of plastics or resins, e.g. aluminized polyester or polyester alone or other impermeable films; and (2) a reservoir layer comprising, for example, a compound of the disclosure in combination with mineral oil, polyisobutylene, and alcohols gelled with USP hydroxymethylcellulose. As another example, the reservoir layer may comprise acrylic-based polymer adhesives with resinous crosslinking agents which provide for diffusion of the active agent from the reservoir layer to the surface of the skin. The transdermal patch may also have a delivery rate-controlling membrane such as a microporous polypropylene disposed between the reservoir and the skin. Ethylene-vinyl acetate copolymers and other microporous membranes may also be used. Typically, an adhesive layer is provided which may comprise an adhesive formulation such as mineral oil and polyisobutylene combined with the active agent.

Other typical transdermal patches may comprise three layers: (1) an outer layer comprising a laminated polyester film; (2) a middle layer containing a rate-controlling adhesive, a structural non-woven material and the active agent; and (3) a disposable liner that must be removed prior to use. Transdermal delivery systems may also involve incorporation of highly lipid soluble carrier compounds such as dimethyl sulfoxide (DMSO), to facilitate penetration of the skin. Other carrier compounds include lanolin and glycerin.

Rectal or vaginal suppositories comprise, for example, an active agent in combination with glycerin, glycerol monopalmitate, glycerol, monostearate, hydrogenated palm kernel oil and fatty acids. Another example of a suppository formulation includes ascorbyl palmitate, silicon dioxide, white wax, and cocoa butter in combination with an effective amount of an active agent.

Nasal spray formulations may comprise a solution of active agent in physiologic saline or other pharmaceutically suitable carder liquids. Nasal spray compression pumps are also well known in the art and can be calibrated to deliver a predetermined dose of the solution.

Aerosol formulations suitable for pulmonary administration include, for example, formulations wherein the active agent is provided in a pressurized pack with a suitable propellant. Suitable propellants include chlorofluorocarbons (CFCs) such as dichlorodifluoromethane, trichlorofluoromethane, or dichlorotetrafluoroethane, carbon dioxide, or other suitable gases. The aerosol may also contain a surfactant such as lecithin. The dose of drug may be controlled by provision of a metered valve.

Dry powder suitable for pulmonary administration include, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. Unit doses for dry powder formulations may be, for example, in the form of capsules or cartridges of, e.g., gelatin, or blister packs from which the powder may be administered by means of an inhaler.

In addition to the foregoing components, it may be necessary or desirable in some cases (depending, for instance, on the particular composition or method of administration) to incorporate any of a variety of additives, e.g., components that improve drug delivery, shelf-life, patient acceptance, etc. Suitable additives include acids, antioxidants, antimicrobials, buffers, colorants, crystal growth inhibitors, defoaming agents, diluents, emollients, fillers, flavorings, gelling agents, fragrances, lubricants, propellants, thickeners, salts, solvents, surfactants, other chemical stabilizers, or mixtures thereof. Examples of these additives can be found, for example, in M. Ash and I. Ash, Handbook of Pharmaceutical Additives (Hampshire, England: Gower Publishing, 1995), the contents of which are herein incorporated by reference.

Appropriate dose and regimen schedules will be apparent based on the present disclosure and on information generally available to the skilled artisan. When the compounds of the disclosure are used in the treatment of HIV, achievement of the desired effects may require weeks, months, or years of controlled, low-level administration of the formulations described herein. Other dosage regimens, including less frequent administration of high-intensity dosages, are also within the scope of the disclosure.

The amount of active agent in formulations that contain the compounds of the disclosure may be calculated to achieve a specific dose (i.e., unit weight of active agent per unit weight of patient) of active agent. Furthermore, the treatment regimen may be designed to sustain a predetermined systemic level of active agent. For example, formulations and treatment regimen may be designed to provide an amount of active agent that ranges from about 0.001 mg/kg/day to about 1000 mg/kg/day for an adult. As a further example, the amount of active agent may range from about 0.1 mg/kg/day to about 500 mg/kg/day, about 0.1 mg/kg/day to about 250 mg/kg/day, about 1 mg/kg/day to about 100 mg/kg/day, about 1 mg/kg/day to about 50 mg/kg/day, or about 1 mg/kg/day to about 25 mg/kg/day. One of skill in the art will appreciate that dosages may vary depending on a variety of factors, including method and frequency of administration, and physical characteristics of the patient.

Treatment regimens that make use of multiple methods of administration are within the scope of the disclosure. For example, when used as smoking cessation agents, a small, steady dose of the compounds of the disclosure may be administered continuously via transdermal patch, while an additional dose can be administered as needed by the patient via chewing gum.

The compounds of the disclosure may be prepared using synthetic methods as exemplified in the experimental section herein, as well as standard procedures that are known to those skilled in the art of synthetic organic chemistry and used for the preparation of analogous compounds. Appropriate synthetic procedures may be found, for example, in J. March, Advanced Organic Chemistry: Reactions, Mechanisms and Structure, 5th Edition (New York: Wiley-Interscience, 2001). Syntheses of representative compounds are detailed in the Examples.

In some embodiments, the compounds of the disclosure are protease inhibitors. Accordingly, the compounds are capable of interfering with the activity of certain proteases, for example HIV protease. In some preferred embodiments, the compounds of the disclosure are equally effective at inhibiting HIV protease in cell free assays and in cell infectivity assays. That is, the presence of cellular matter does not reduce the efficacy of the compounds. In some other embodiments, the compounds of the disclosure exhibit a modest decrease in efficacy between a cell free assay and a cell infectivity assay. For example, the IC₅₀ values of the compounds in a cell infectivity assay are no more than 100% greater than the IC₅₀ values of the compounds in a cell free assay, or no more than 50% greater, or no more than 25% greater, or no more than 10% greater. In some embodiments, the IC₅₀ values of the compounds in a cell infectivity assay are less than the IC₅₀ values of the compounds in a cell free assay. In some preferred embodiments, the compounds of the disclosure exhibit IC₅₀ values in cell infectivity assays that are below about 75 nM, or below about 50 nM, or below about 25 nM, or below about 10 nM.

Accordingly, the compounds find utility in treating viral infections. In certain embodiments, the compounds are useful as inhibitors of HIV protease. The compounds of the disclosure, and compositions comprising such compounds, are useful in the treatment of AIDS or HIV infections, including multidrug-resistant strains of HIV.

Accordingly, the disclosure provides a method for treating an HIV-infected patient, the method comprising administering to the patient an effective amount of any of the compounds disclosed herein. The disclosure also provides a method for preventing viral replication, the method comprising administering an effective amount of any of the compounds disclosed herein. The disclosure also provides a method for inhibiting the activity of HIV-1 protease, the method comprising administering an effective amount of any of the compounds disclosed herein. The disclosure also provides a method for treating a patient suffering from AIDS, the method comprising administering an effective amount of any of the compounds disclosed herein. The disclosure also provides a method for inhibiting the spread of HIV-virions to non-infected cells, the method comprising contacting a cell infected with HIV with an effective amount of any of the compounds disclosed herein. As described in more detail herein, in any of the aforementioned methods, the compound may be administered in a composition comprising one or more active agents and one or more additives.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

It is to be understood that while the invention has been described in conjunction with the preferred specific embodiments thereof, that the foregoing description as well as the examples that follow, are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains.

### EXAMPLES

In the Examples that follow, references are made to conjugate compounds that include “derivatives” of Atazanavir, Saquinavir, etc., or “cores” of Atazanavir, Saquinavir, etc. It will be understood that these references are used for ease of description only. Thus, an “Atazanavir conjugate” or a conjugate that comprises an “Atazanavir derivative” or an “Atazanavir core” are references to a compound wherein a fragment of the compound is derived from (or resembles) Atazanavir per se. For example, in a simple embodiments, an Atazanavir conjugate may have the Atazanavir structure per se with the exception that one of the atoms on the structure has been replaced with a linkage to one of the linker moieties (or second moieties) described herein. In another example, an Atazanavir conjugate may have the Atazanavir structure with multiple exceptions that may, for example, be selected from the following: one of the atoms is replaced with a linkage to the linker/second moiety, one of the functional groups is protected with a protecting group, one of the functional groups has been replaced with an alternate functional group, etc.

### Example 1

### Preparation of Atazanavir Derivative Conjugate

Scheme 1 below shows the synthesis of a conjugated based on an atazanavir derivate core. The synthesis is convergent, and begins with an atazanavir derivative core. Reaction with N-t-Boc-L-tert-Leucine by coupling with the HATU reagent yields the orthogonally-protected atazanavir, with the left side amine protected as a t-Boc derivative, and the right side amine protected as a benzyl carbamate derivative. Benzyl removal (mild hydrogenolysis) exposes the right side amine, which is then reacted with the SLFA 2 fragment that has been activated by the reaction with 4-hydroxybutanoic acid anhydride, followed by treatment of the exposed alcohol with carbonyl-diimidazole. Coupling of these two fragments is followed by replacement of the tert-Boc group with methoxycarbonyl that is present in Atazanavir.

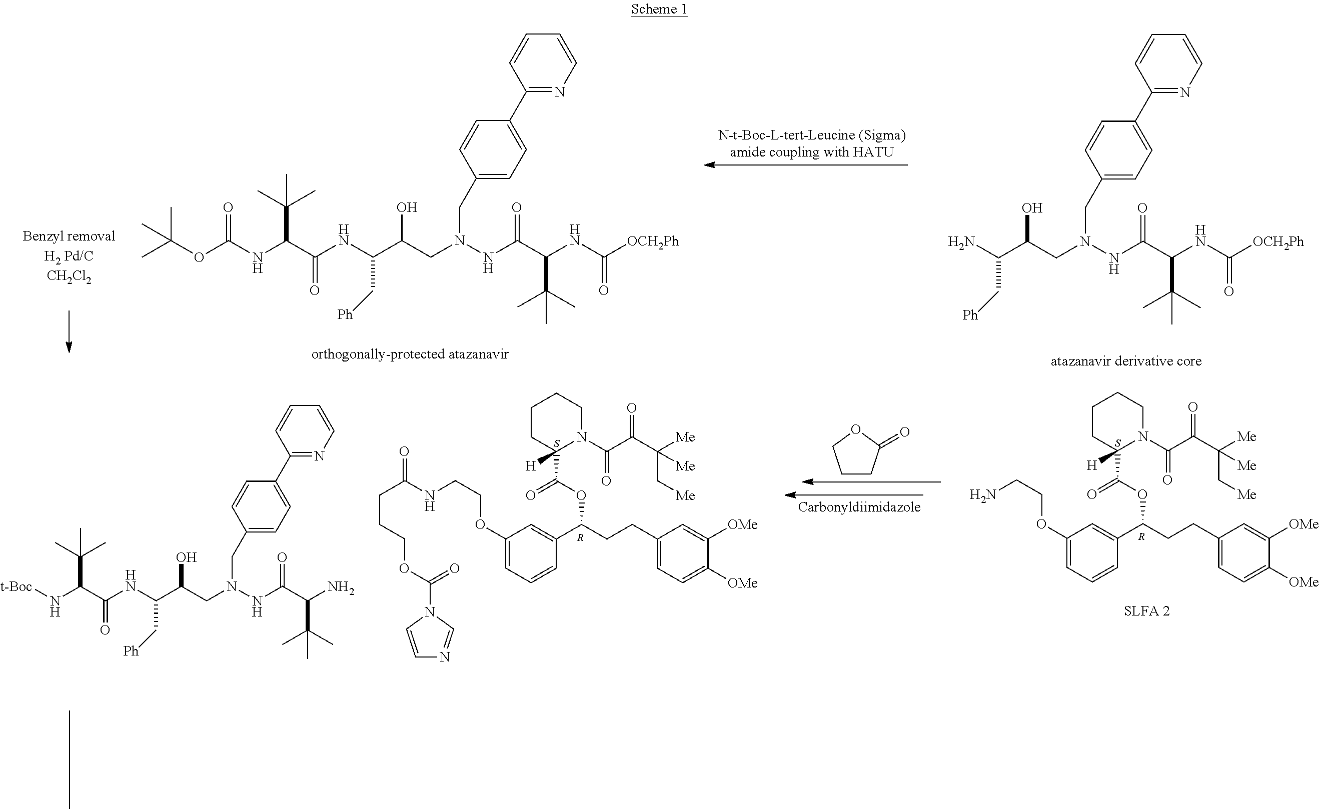

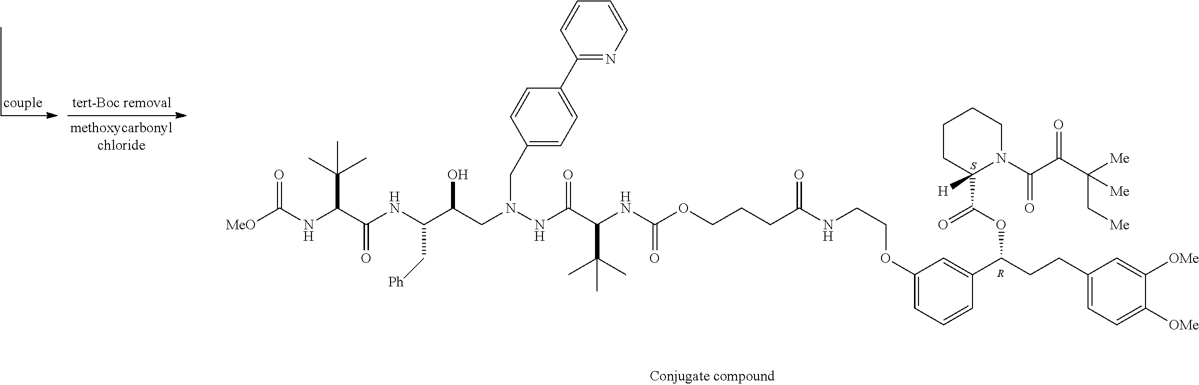

The final conjugate has an SLF portion and a protease inhibitor. Scheme 2 below shows the same approach to Atazanavir, but coupled with SLF 1 hydroxyl. The core of Atazanavir is prepared and then coupled, as above, with the protected L-leucine derivative, but in this case, the “left side” amine is present in the desired final form (i.e., the methoxy-carbamate present in Atazanavir). Removal of the tert-Boc group is followed by coupling with the SLF-1 hydroxyl derivative that has been activated by treatment with carbonyl-diimidazole to yield the Atazanavir derivative-SLF1 carbamate conjugate.

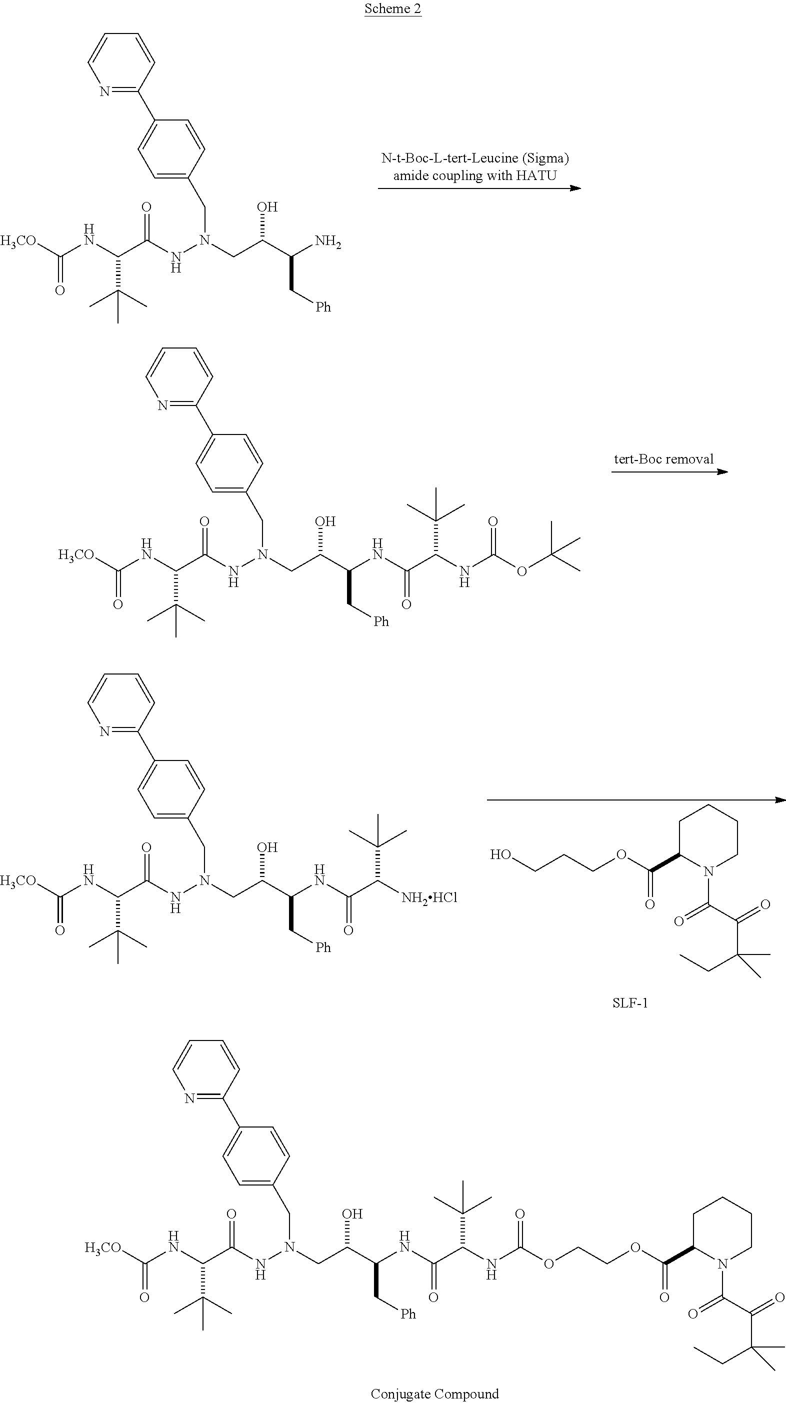

### Example 2

### Preparation of Amprenavir Derivative Conjugate

The synthesis of an Amprenavir derivative carbamate conjugate with SLF-2 is shown in Scheme 3 below. The SLF-2 fragment is acylated with 4-hydroxybutanoic acid anhydride. The hydroxyl group is then activated with carbonyl diimidazole to give the activated SLF-2 fragment. This fragment is reacted with the Amprenavir derivative core to yield the Amprenavir Derivative-SLF-2 carbamate.

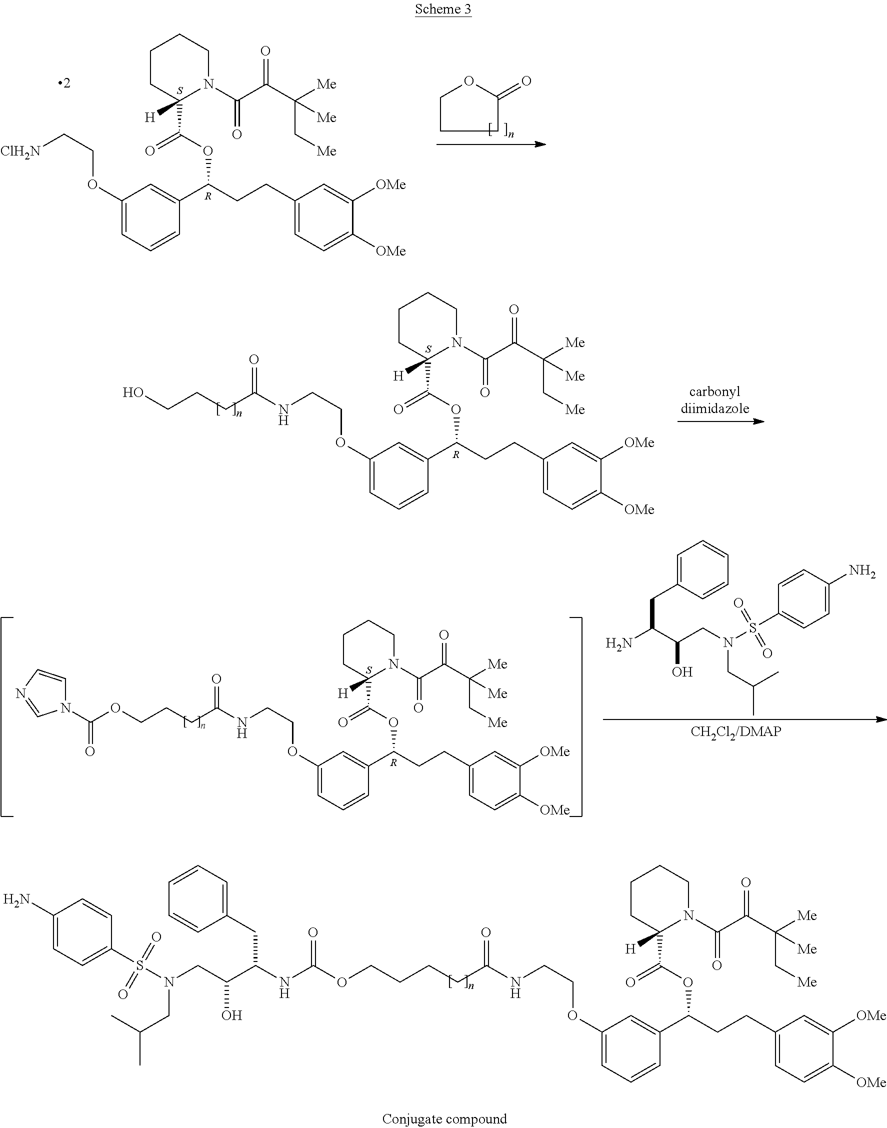

The Amprenavir core with the carbamate linker is the portion on the left side of the molecule, whereas the SLF-2 fragment is the portion on the right. The chemistry is based on the formation of a stable carbamate.

### Example 3

### Preparation of Lopinavir Derivative Conjugate

The synthesis is shown in Scheme 4 below. The starting material is benzylated ureidol-L-leucine. The material is reacted with the CDI-activated SLF 2 carbamate core (see previous examples). Hydrogenolytic debenzylation of this molecule provides the ureido-L-leucyl in the de-protected carboxylate form. This is coupled with the amine fragment. The Lopinavir derivative SLF-2 carbamate conjugate is shown in the Scheme.

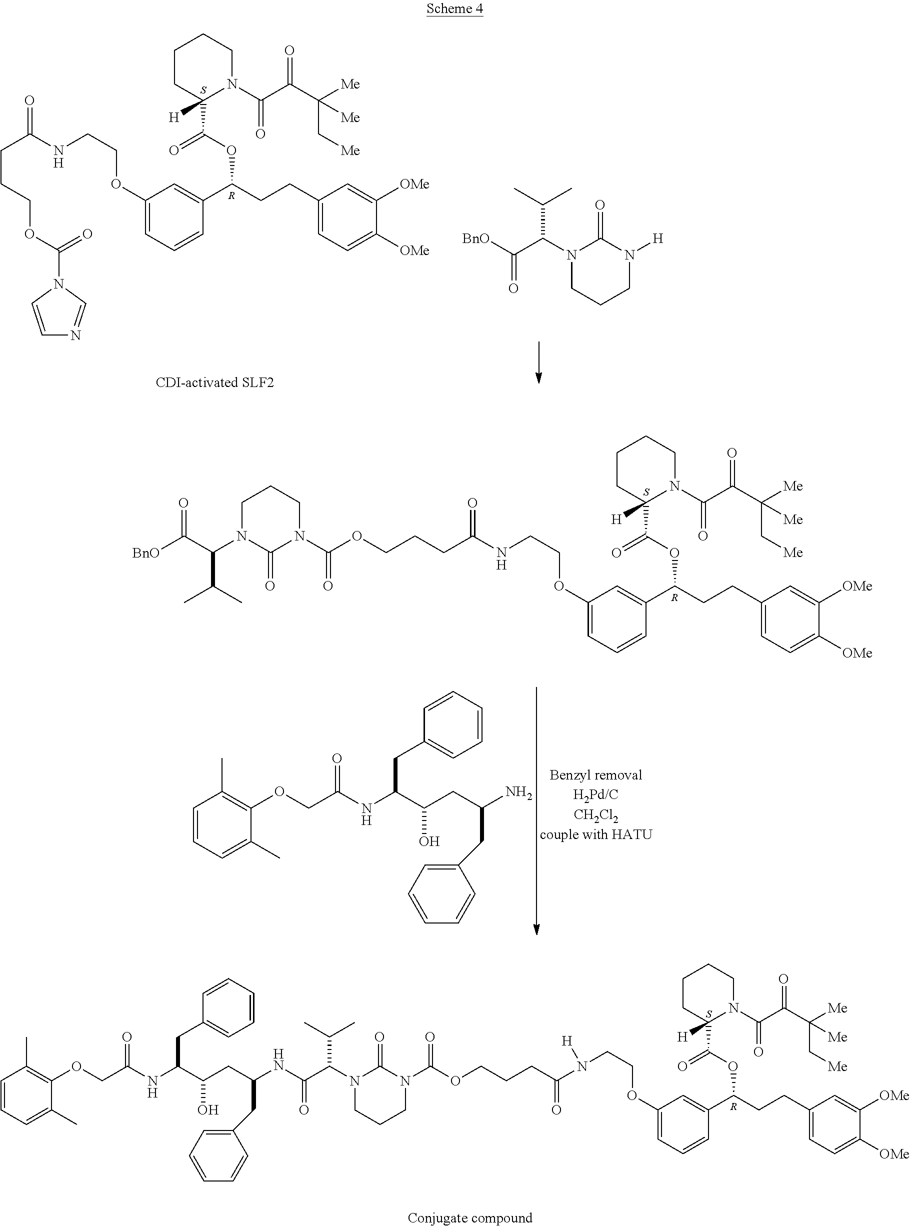

In some embodiments, the SLF 2 fragment may be activated using a simple alkyl linkage directly to the ureido-L-leucyl fragment, as shown in Scheme 5 below.

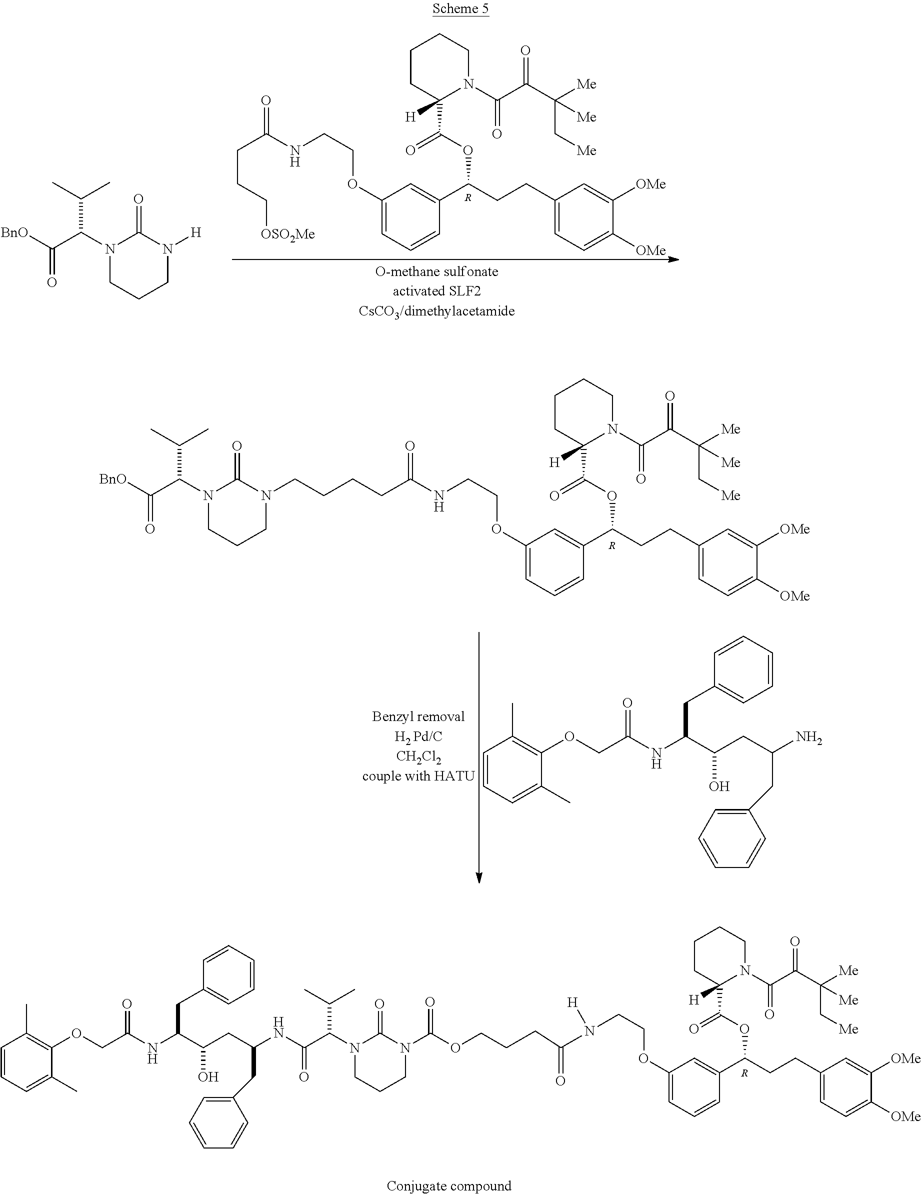

### Example 4

### Preparation of Saquinavir Derivative Carbamate Conjugates

Two approaches may be taken to preparing two separate Saquinavir conjugates, as shown in Scheme 6 below. A Saquinavir derivative core may be used as starting material. In one permutation, the quiniline “side” of Saquinavir may be modified starting with N-t-Boc-8-amino quinaldic acid through simple HATU coupling of the amino group of the Saquinavir core and the carboxylic acid of the quinaldic acid. This intermediate is subjected to t-Boc removal under standard conditions, and is then coupled with the CDI-activated SLF 2 to yield the Squinavir derivative SLF-2 8-aminoquinaldic acid carbamate conjugate shown.

In a second permutation, also shown in Scheme 6, the Saquinavir derivative core is reacted similarly as described above with 5-N-t-Boc-2-carboxy pyridine. Removal of the boc group and coupling with CDI-activated SLF 2 gives the alternative Saquinavir derivative SLF-2 pyridiyl carbamate conjugate shown.

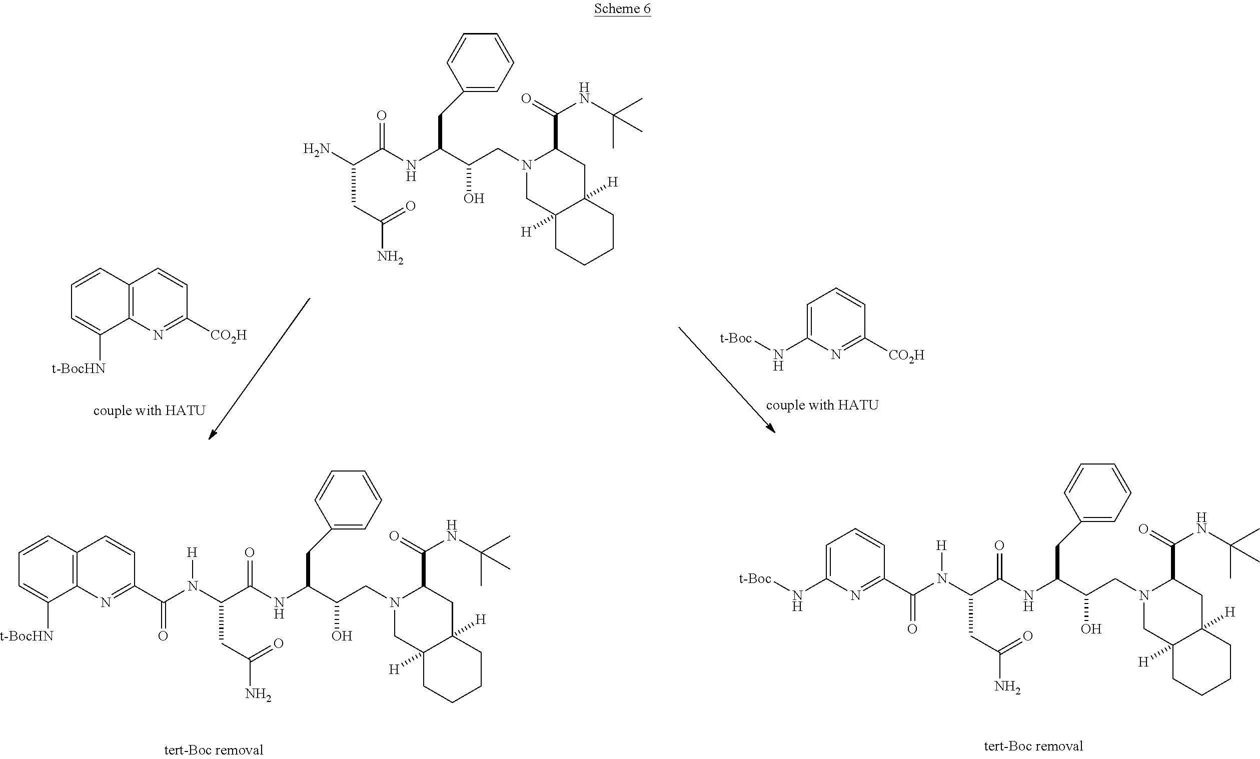

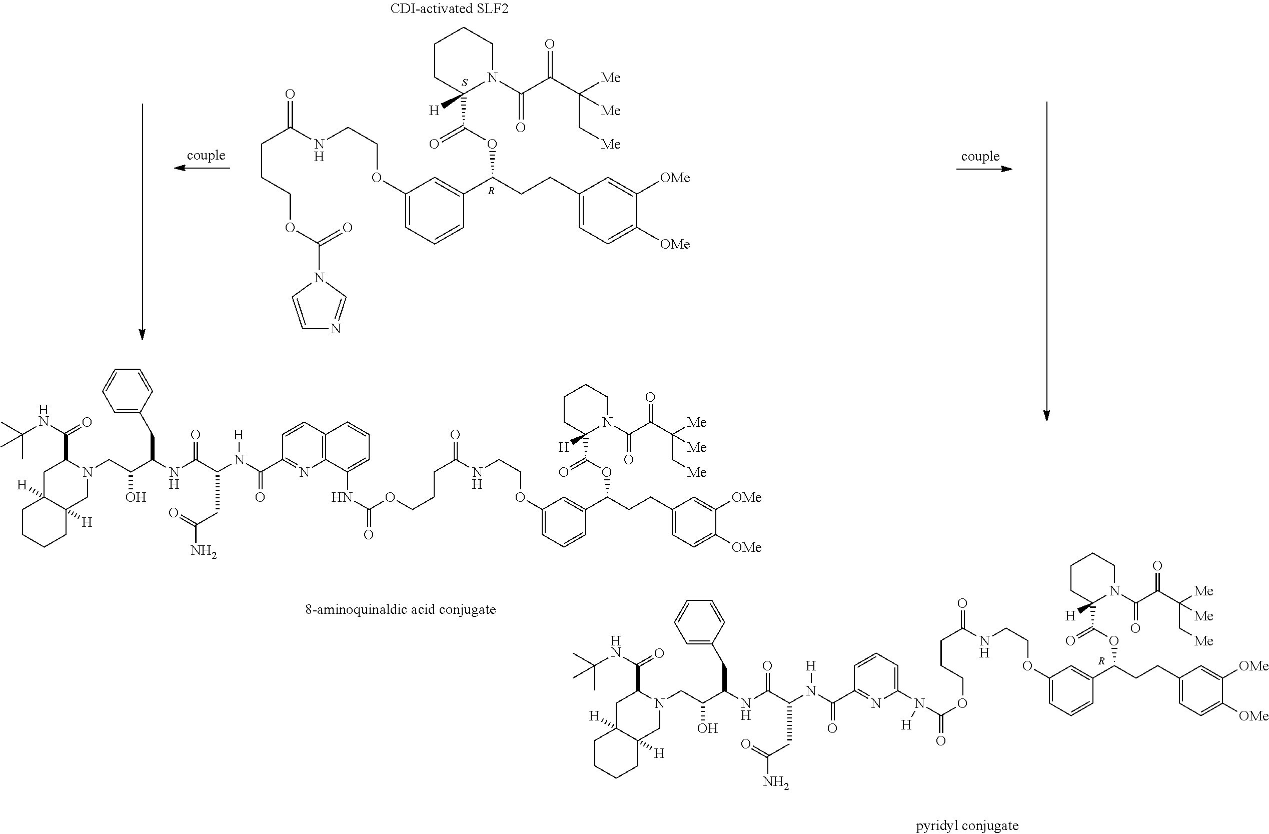

### Example 5

### Preparation of FK506-Atazanavir Conjugate

Acrylic acid-modified FK506 is used to couple with the Atazanavir core as shown in Scheme 7 below. The Atazanavir core is used as prepared previously (described above). This is a simple one-step procedure that yields the benzyloxy-carbonyl protected Atazanavir. This conjugate is tested for activity, and is further modified by removal of the benzyl group and reaction with methoxy-carbonyl chloride to give the methoxy-carbamate group present in the native Atazanavir protease inhibitor molecule.

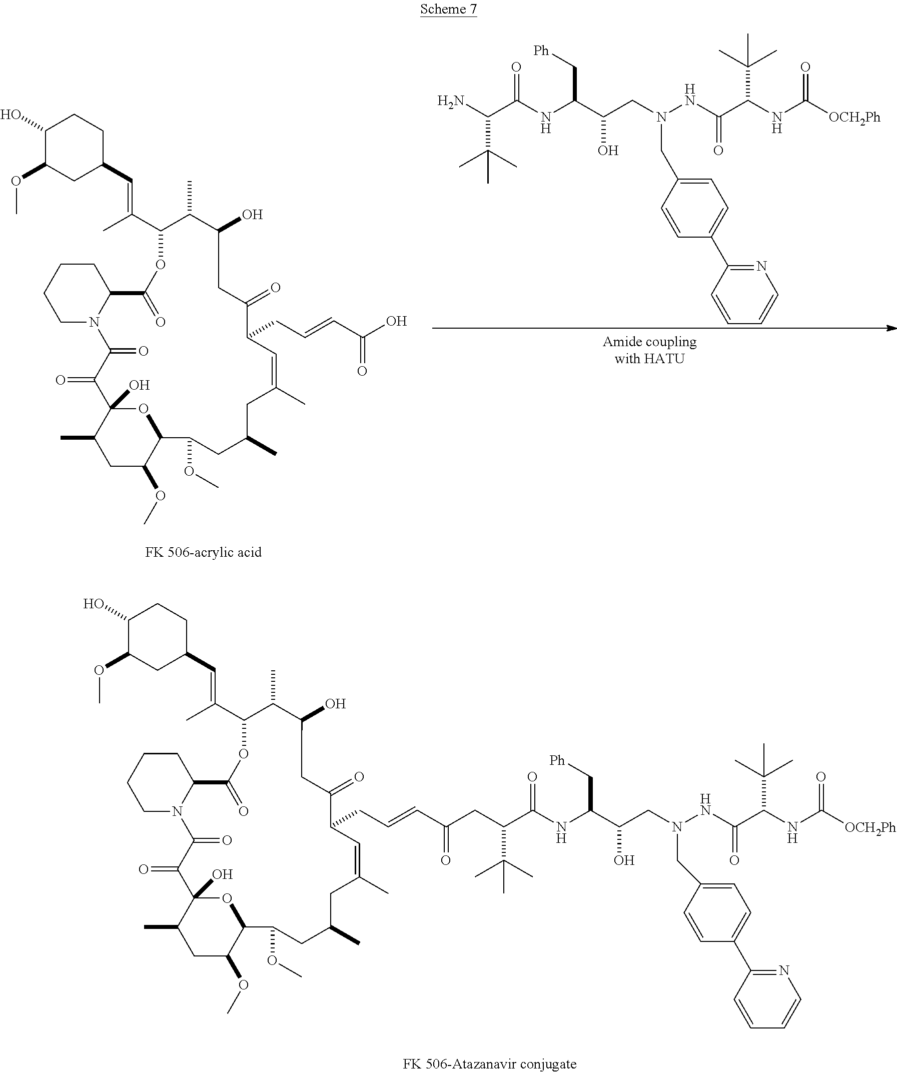

### Example 6

### Preparation of Amprenavir Derivative Furanoyl Conjugate

Here is provided a synthesis of a furanose linker that retains the hydroxylation present on the starting material for the synthesis, namely diacetone glucose. This synthesis is shown in Scheme 8 below. Solubility issues can be overcome by increasing the solvent exposure of the molecule by virtue of the incorporation of the hydroxyl groups. The SLF 1 conjugate is shown in the Scheme, but this chemistry is not limiting and can be adapted to fit any of the SLFs.

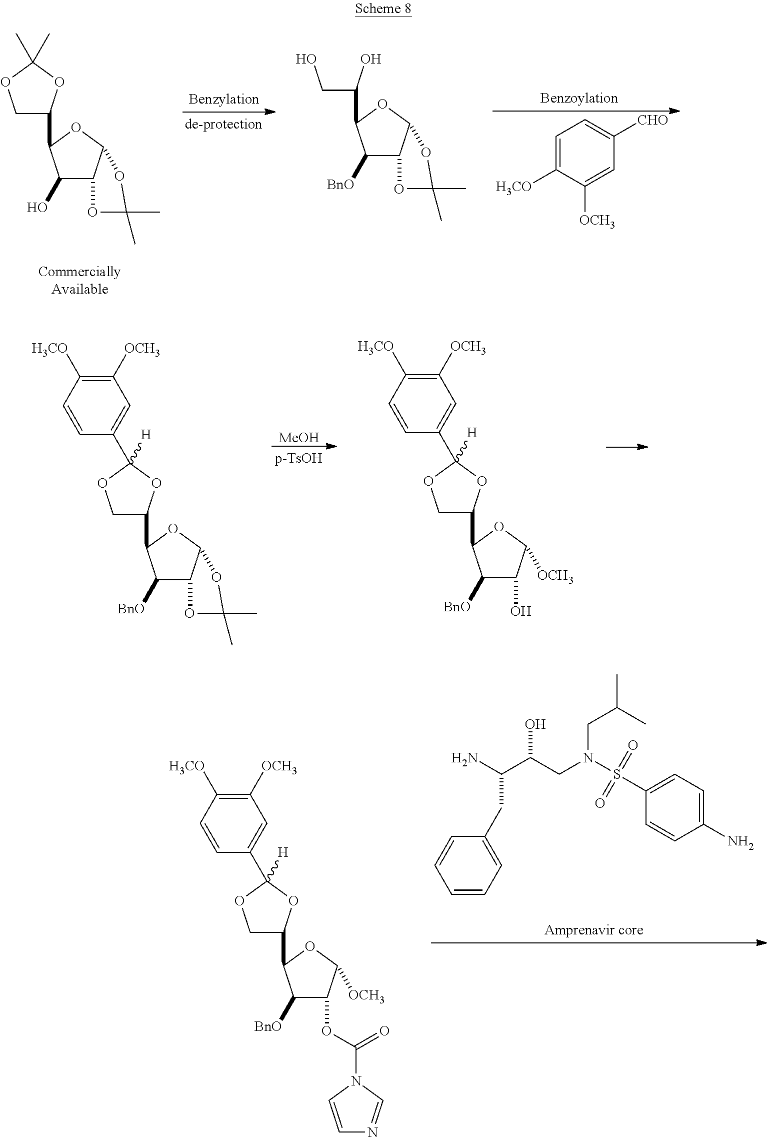

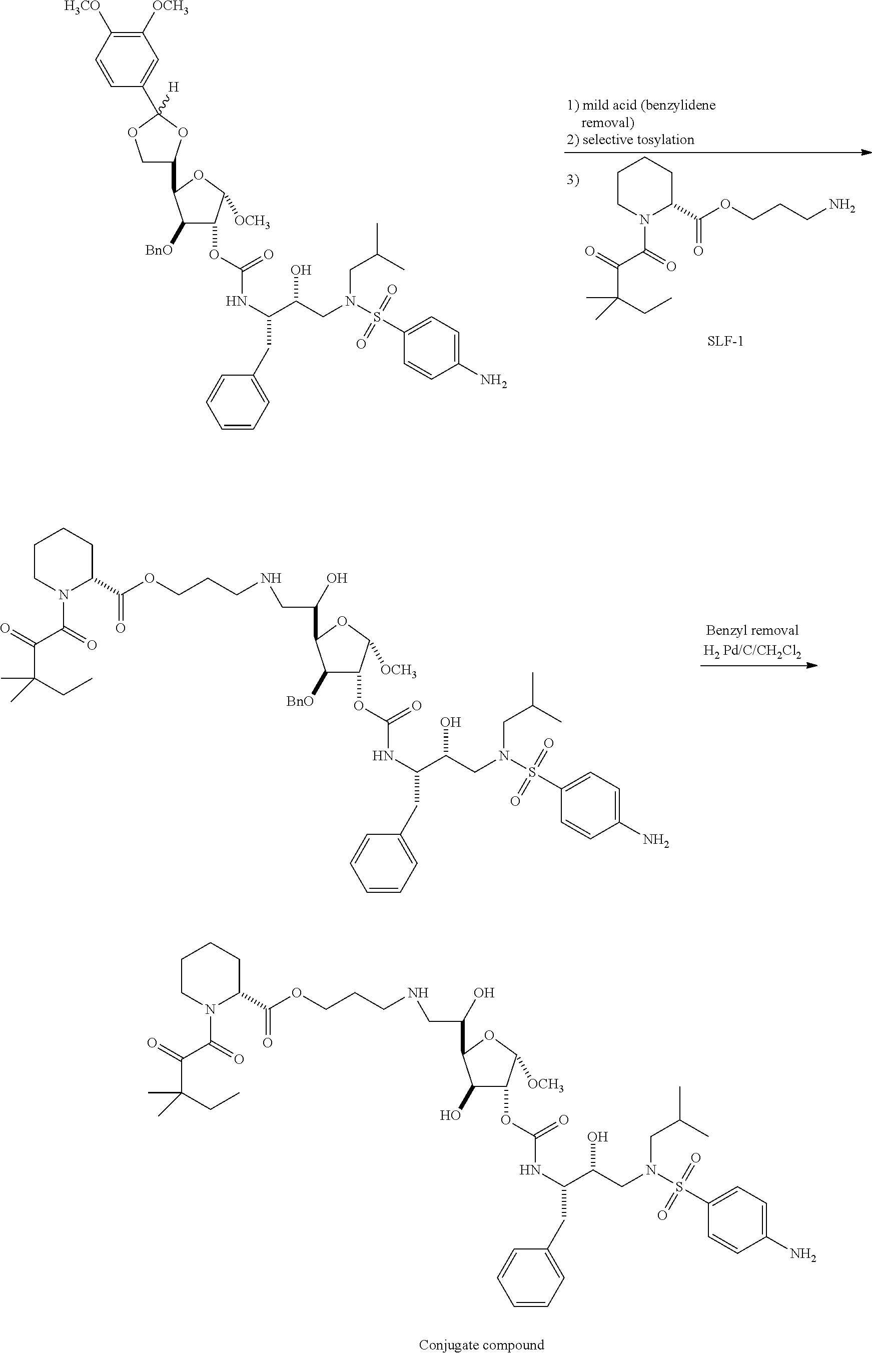

### Example 7

### Preparation of Darunavir-SLF Conjugate

Scheme 9 below details the synthesis of Darunavir conjugates. A Darunavir analogue containing a hydroxymethyl-aryl group has antiviral activity (IC₉₀=1.8 nM) with a binding constant measured in the low picomolar range. All four SLF conjugates of Darunavir can be prepared; two of these, the SLF 1 and SLF 2 conjugates, are shown in Scheme 9

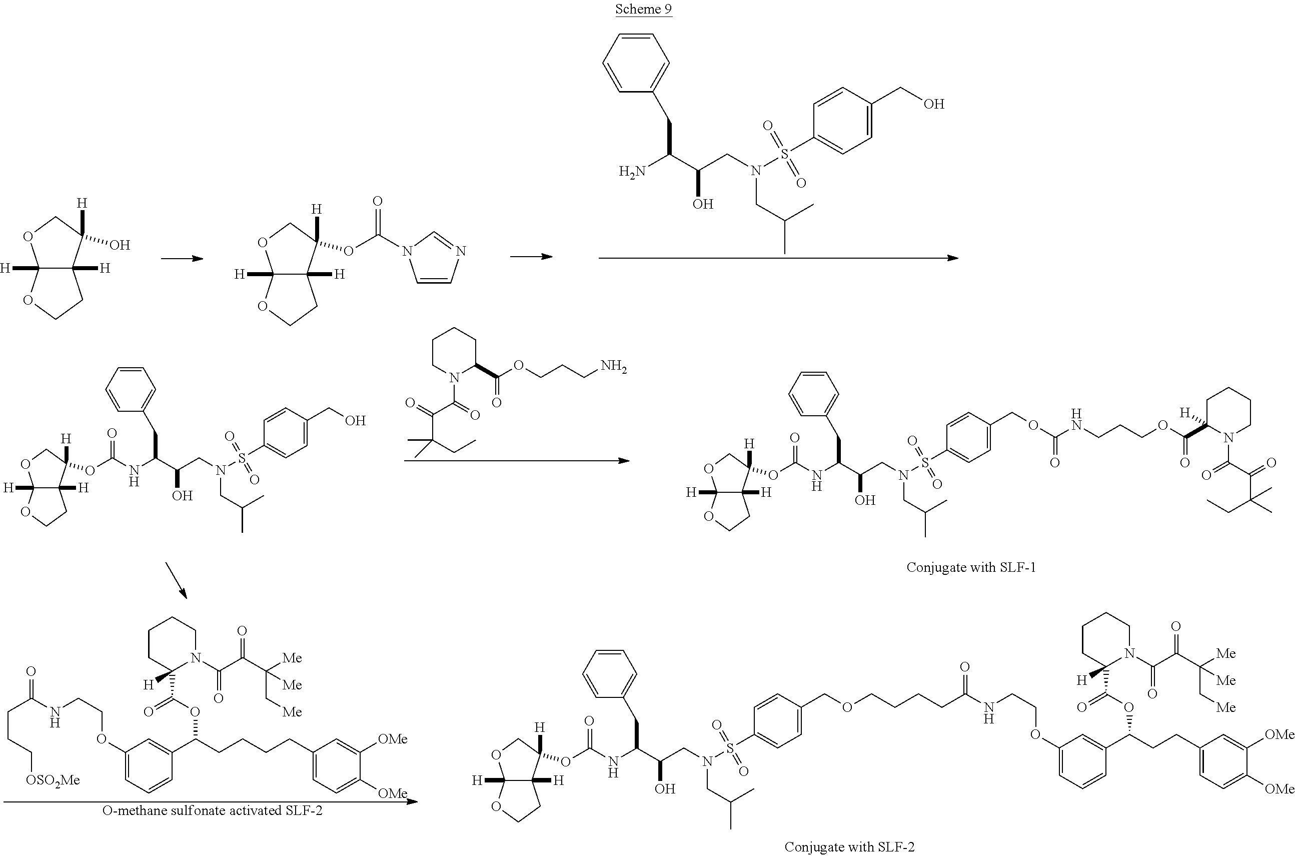

### Example 8

Further examples of compounds according to the disclosure were prepared as shown in the following Schemes. An Atazanavir derivative conjugate with SLF-2 is shown in Scheme 10. Another Atazanavir derivative conjugate with SLF-2 is shown in Scheme 11.

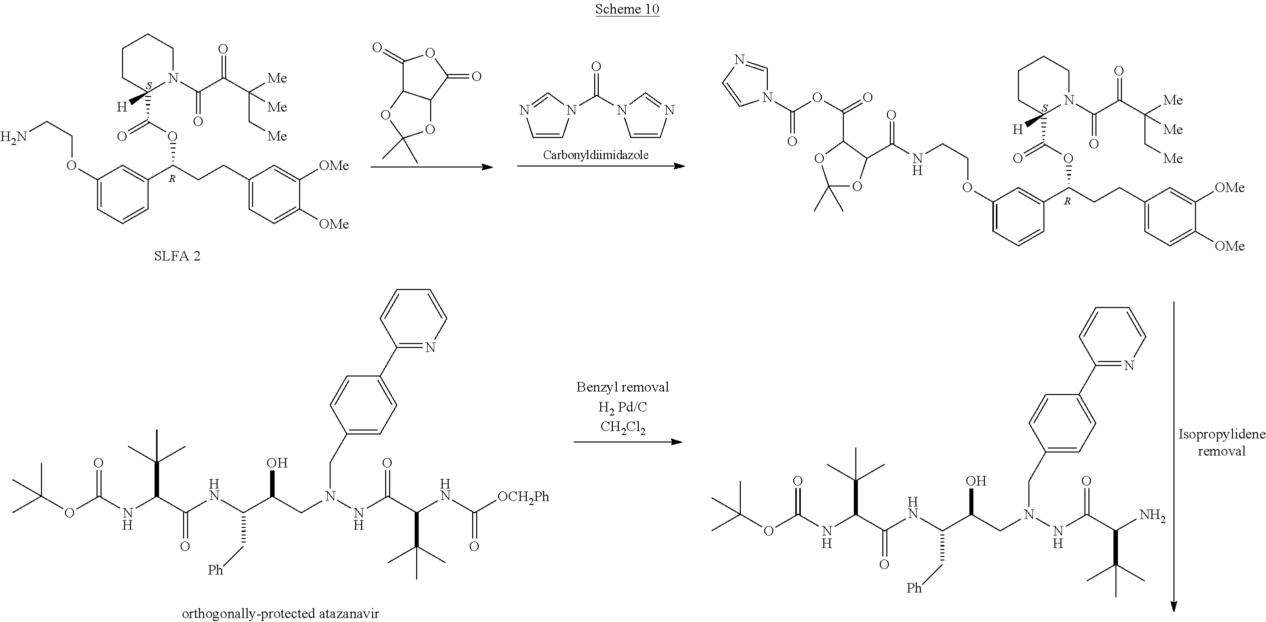

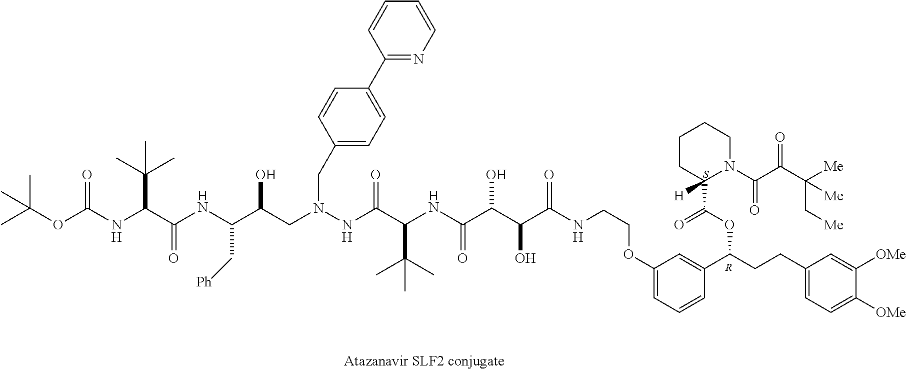

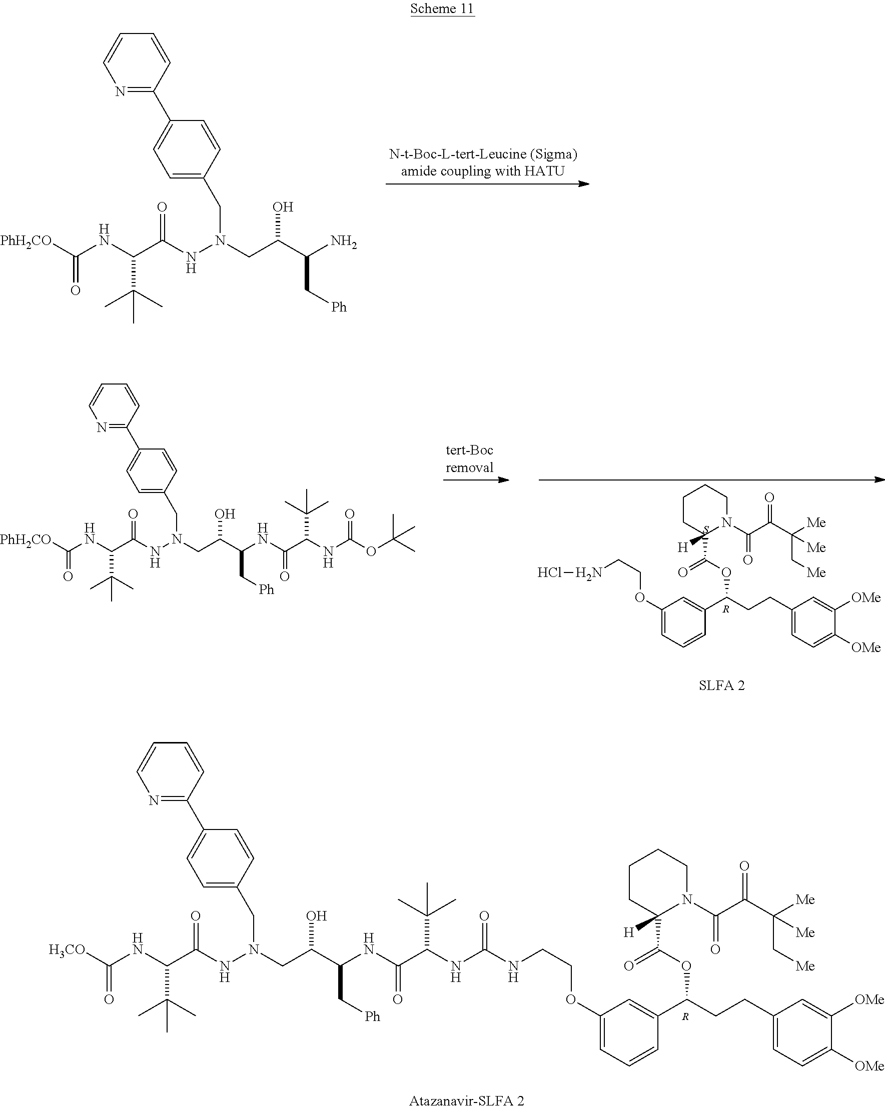

### Example 9

### Cell Free and Cell Infectivity Assays

Procedure—cell free assay. Cell free assays may be conducted according to the test kit protocol provided with the SensoLyte™ 490 HIV-1 Protease Assay Kit, available from AnaSpec (San Jose, Calif.).

Procedure—cell infectivity assay. The T-cell-tropic strain HIV-1LAI may be used to infect CEM-T4 cells over a dose range of the protease inhibitor compounds. CEM-T4 cells may be grown in RPMI 1640 medium supplemented with 10% heat-inactivated fetal bovine serum, penicillin (100 units/mL), streptomycin (100 ug/mL), and polybrene (2 ug/mL) at 37° C. with 5% CO2. The PI dose range covers a total of nine 3:1 dilutions from 5000 nM to less than 1 nM. The titered virus is added to wells of CEM cells (pre-treated with protease inhibitor [PI] dilution series for 1 h) at a low multiplicity of infection (MOI=0.01) and incubated for 4 h at 37° C. The cells are washed three times with PBS (GIBCO/BRL), resuspended in triplicate wells each with 1 ml of culture medium containing the same concentration of PI as the initial pre-incubation and further incubated at 37° C. in 5% CO₂. Each well of the 24-well plate contains 1×10̂5 cells upon incubation initiation. The cells are fed every two days with fresh PI at the appropriate concentration. Samples are scored for cytopathic effect on day 4, and if consistent cytopathic effect (CPE) was seen throughout the various wells, the supernatants are collected for p24 assay. Otherwise, the cultures are maintained with feeding every other day until day 8, at which point the p24 are initiated.

Using the above procedures, the IC₅₀ values of compounds according to the disclosure may be obtained in cell free assays and cell infectivity assays. The values may be compared with the IC₅₀ values for Amprenavir and Lopinavir.

What is claimed is:

1. A compound having the structure of formula (I) 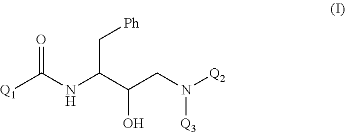
wherein:
1 is substituted heteroatom-containing alkyl, optionally substituted with -L-U, or Q¹ is cycloalkoxy;
Q² is arylsulfonyl substituted with -L-U, or Q² is alkylamido optionally substituted with -L-U, provided that either Q¹ or Q² is substituted with -L-U;
Q³ is alkyl or aralkyl, or wherein Q² and Q³ are taken together to form a cyclic group;
L is a linking moiety;
U is selected from Unit A, Unit B, and Unit C 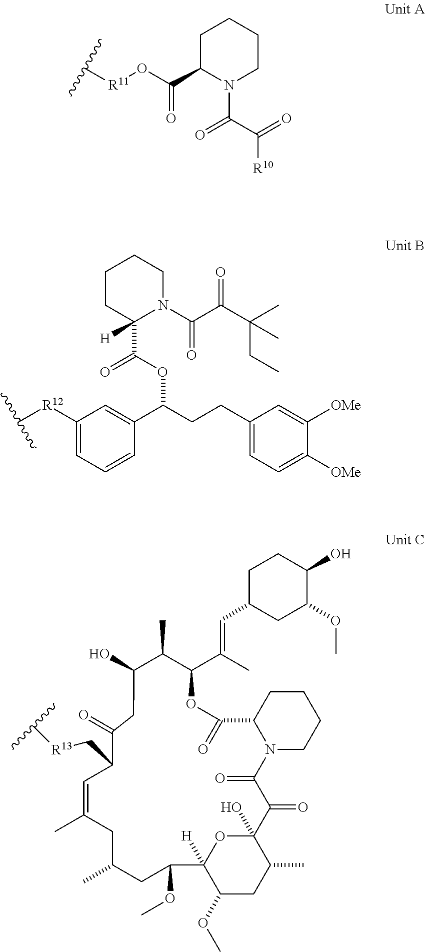
wherein:
the wavy line represents the attachment point to the remainder of the compound;
R¹⁰ is selected from 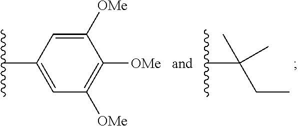
R¹¹ is selected from a bond, —(CH₂)ₙ₁—, —(CH₂)ₙ₁—O—, and —(CH₂)ₙ₁—NH—, where n1 is an integer from 1 to 5 (that is, 1, 2, 3, 4, or 5);
R¹² is selected from a bond, —(CH₂)ₙ₂—, —(CH₂)ₙ₂—O—, and —O—(CH₂)ₙ₂—NH—, where n2 is an integer from 1 to 5 (that is, 1, 2, 3, 4, or 5); and
R¹³ is selected from a bond, —(CH₂)ₙ₃—, —(CH₂)ₙ₃—O—, and —(CH₂)ₙ₃—NH—, where n3 is an integer from 1 to 5 (that is, 1, 2, 3, 4, or 5),
or a stereoisomer, salt, or prodrug thereof.

2. The compound of claim 1, wherein:
Q¹ is selected from —CR^{a}—X⁴, —O—Y¹, and —CR^{b}—NH—C(═O)—Z¹—NH—C(═O)—O-L-U, wherein R^{a} is alkyl and R^{b} is —CH₂—C(═O)—NH₂;
Q² is selected from —NH—C(═O)—CR^{a}—NH—C(═O)—O—X⁵ and —SO₂—Ar¹-L-U, wherein Ar¹ is an optionally substituted phenylene;
Q³ is selected from —CH₂—Ar¹—Ar² and alkyl, wherein Ar² is optionally substituted pyridyl, or wherein Q² and Q³ are linked to form a cycle;
X⁴ is an amide, a carbamate, or -L-U;
X⁵ is alkyl, aryl, aralkyl, alkaryl, or -L-U;
Y¹ is a heterocyclic group, with the proviso that, when Q² is —SO₂—Ar¹-L-U and Q³ is alkyl, then Y¹ is not tetrahydrofuranyl; and
Z¹ is an arylene group which may be substitute or unsubstituted, and which may contain one or more heteroatoms.

3. The compound of claim 2, wherein:
R^{a} is t-butyl;
X⁴ is —NH—C(═O)—OCH₃;
X⁵ is methyl or benzyl;
Y¹ comprises two or more fused rings and two or more heteroatoms;
Z¹ is pyridylene or quinolinylene;
n1 is 2 or 3;
n2 is 2 or 3; and
n3 is 2 or 3.

4. The compound of claim 2, wherein:
Y¹ is bis(tetrahydrofuranyl);
Ar¹ is phenylene; and
Ar² is pyridyl.

5. The compound of claim 1, wherein the compound has the structure of formula (Ia) 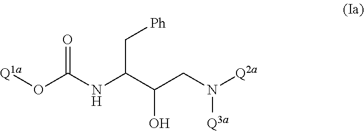
wherein:
Q^{1a} is a cyclic group optionally comprising two or more fused rings and optionally heteroatom-containing;
Q^{2a} is —SO₂—Ar¹-L-U, wherein Ar¹ is an optionally substituted phenylene; and
Q^{3a} is alkyl.

6. The compound of claim 1, wherein the compound has the structure of formula (Ib) 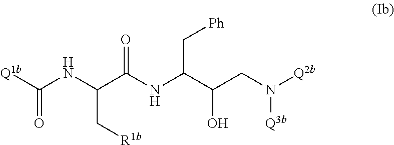
wherein:
Q^{1b} is —Z¹—NH—C(═O)-L-U, wherein Z¹ is an arylene group which may be substitute or unsubstituted, and which may contain one or more heteroatoms;
Q^{2b} and Q^{3b} are linked, together with the nitrogen atom to which they are attached, to form a heterocyclic ring system which optionally comprises 2 or more fused rings; and
R^{1b} is substituted or unsubstituted carbamoyl.

7. The compound of claim 1, wherein the compound has the structure of formula (Ic) 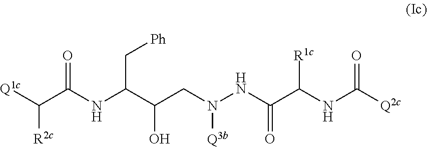
wherein
Q^{1c} is selected from -L-U, alkylamido, and —NH—C(═O)—O-L-U;
Q^{2c} is selected from —O-L-U and —O—R^{3c};
Q^{3c} is aralkyl which is optionally heteroatom-containing;
R^{1c} and R^{2c} are individually selected from alkyl groups; and
R^{3c} is selected from alkyl, aryl, alkaryl, and aralkyl.

8. The compound of claim 1, wherein the linker moiety is selected from a bond, alkylene, alkenylene, alkynylene, arylene, aralkylene, and alkarylene, any of which may be substituted or unsubstituted, and any of which may contain one or more heteroatoms.

9. The compound of claim 8, wherein the linker moiety is selected from substituted or unsubstituted heteroalkylene, heteroarylene, alkylenecarbonyl, arylenecarbonyl, alkyleneoxycarbonyl, aryleneoxycarbonyl, alkylenecarbonato, arylenecarbonato, alkylenecarbamoyl, arylcarbamoyl, alkyleneamine, aryleneamine, alkyleneamide, and aryleneamide.

10. The compound of claim 1, wherein the compound has an IC₅₀ value in a cell infectivity assay that is no more than twice the IC₅₀ value in a cell infectivity assay.

11. The compound of claim 1, wherein the compound has an IC₅₀ value in a cell infectivity assay that is no more than 50 nM.

12. A pharmaceutical formulation comprising the compound of claim 1 and a pharmaceutically acceptable carrier.

13. The pharmaceutical formulation of claim 12, further comprising one or more additives and optionally comprising one or more additional active agents.

14. A method for treating a patient with a protease inhibitor comprising administering an effective amount of the compound of claim 1 to a patient in need thereof.

15. The method of claim 14, wherein the patient is infected with HIV.

16. The method of claim 14, wherein the patient is suffering from AIDS.

17. The method of claim 15, wherein the HIV is multiple-drug resistant HIV.

18. A compound having a structure selected from formulae (II), (III), (IVa), and (IVb) 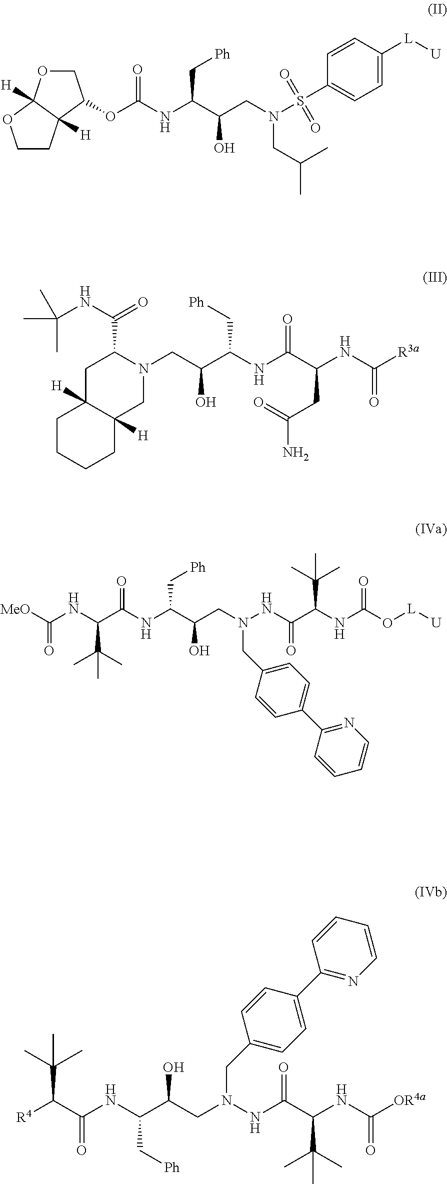
wherein:
R^{3a} is selected from 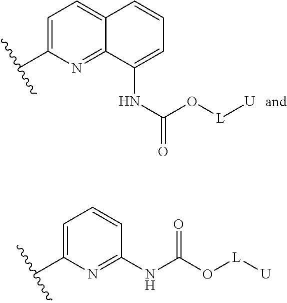
R⁴ is selected from —NH—C(═O)—O-L-U and -L-U;
R^{4a} is selected from alkyl, aryl, alkaryl, and aralkyl;
L is a linking moiety;
U is selected from Unit A, Unit B, and Unit C 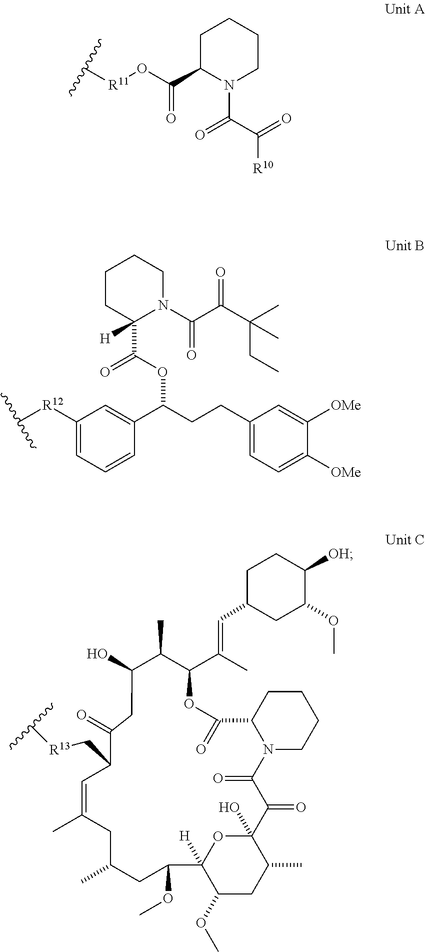
wherein:
the wavy line represents the attachment point to the remainder of the compound;
R¹⁰ is selected from 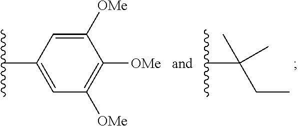
R¹¹ is selected from a bond, —(CH₂)ₙ₁—, —(CH₂)ₙ₁—O—, and —(CH₂)ₙ₁—NH—, where n1 is an integer from 1 to 5 (that is, 1, 2, 3, 4, or 5);
R¹² is selected from a bond, —(CH₂)ₙ₂—, —(CH₂)ₙ₂—O—, and —O—(CH₂)ₙ₂—NH—, where n2 is an integer from 1 to 5 (that is, 1, 2, 3, 4, or 5); and
R¹³ is selected from a bond, —(CH₂)ₙ₃—, —(CH₂)ₙ₃—O—, and —(CH₂)ₙ₃—NH—, where n3 is an integer from 1 to 5 (that is, 1, 2, 3, 4, or 5),
or a stereoisomer, salt, or prodrug thereof.

19. A method for inhibiting the action of HIV-1 protease, the method comprising administering a conjugate compound comprising: (1) a core selected from atazanavir, saquinavir, darunavir, and analogs or derivatives thereof; (2) a linking moiety; and (3) a second moiety capable of binding to a FK506-binding protein.

20. The method of claim 19, wherein the compound has the structure of formula (I) 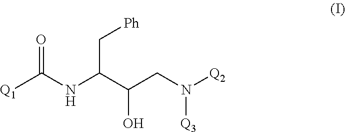
wherein:
Q¹ is substituted heteroatom-containing alkyl, optionally substituted with -L-U, or Q¹ is cycloalkoxy;
Q² is arylsulfonyl substituted with -L-U, or Q² is alkylamido optionally substituted with -L-U, provided that either Q¹ or Q² is substituted with -L-U;
Q³ is alkyl or aralkyl, or wherein Q² and Q³ are taken together to form a cyclic group;
L is a linking moiety;
U is selected from Unit A, Unit B, and Unit C 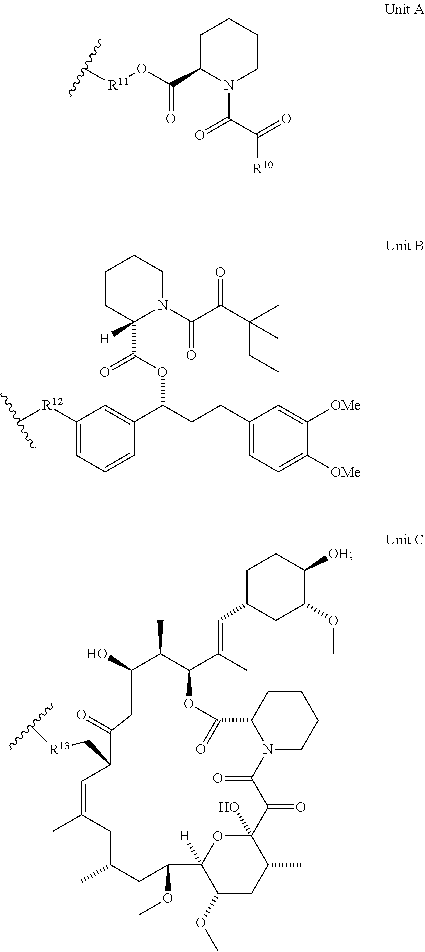
wherein:
the wavy line represents the attachment point to the remainder of the compound;
R¹⁰ is selected from 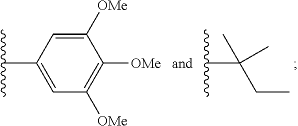
R¹¹ is selected from a bond, —(CH₂)ₙ₁—, —(CH₂)ₙ₁—O—, and —(CH₂)ₙ₁—NH—, where n1 is an integer from 1 to 5 (that is, 1, 2, 3, 4, or 5);
R¹² is selected from a bond, —(CH₂)ₙ₂—, —(CH₂)ₙ₂—O—, and —O—(CH₂)ₙ₂—NH—, where n2 is an integer from 1 to 5 (that is, 1, 2, 3, 4, or 5); and
R¹³ is selected from a bond, —(CH₂)ₙ₃—, —(CH₂)ₙ₃—O—, and —(CH₂)ₙ₃—NH—, where n3 is an integer from 1 to 5 (that is, 1, 2, 3, 4, or 5),
or a stereoisomer, salt, or prodrug thereof.

21. The method of claim 19, wherein the second moiety and linker are attached to the arylsulfonyl group of darunavir or a derivative thereof, or wherein the second moiety and linker are attached to the heteroaryl group of saquinavir or a derivative thereof.
